(19) United States
(12) Patent Application Publication
(10) Pub. No.: US 2017/0336397 A1
(43) Pub. Date: Nov. 23, 2017

(54) RFID Detection Systems And Methods

(71) Applicant: Roche Molecular Systems, Inc., Pleasanton, CA (US)

(72) Inventors: Stanford Kwang, Pleasanton, CA (US); Wouter Pattje, Livermore, CA (US)

(73) Assignee: Roche Molecular Systems, Inc., Pleasanton, CA (US)

(21) Appl. No.: 15/598,163

(22) Filed: May 17, 2017

Related U.S. Application Data
(60) Provisional application No. 62/338,618, filed on May 19, 2016.

(51) Int. Cl. G01N 33/53; G06K 19/07

(52) U.S. Cl. CPC G01N 33/5308; G06K 19/0723

(57) ABSTRACT

Improved detection and tracking methods and systems are disclosed herein. The use of RFID labels that are operably connected upon complex formation in a binding assay is described as well as the use of RFID labels and supplemental identifiers to enhance component tracking in assay systems.

### CROSS-REFERENCE TO RELATED APPLICATION

The present application claims the benefit of U.S. Provisional Patent Application Ser. No. 62/338,618, filed May 19, 2016. Reference is also made to U.S. Provisional Patent Application Ser. No. 62/338,620, filed May 19, 2016, and U.S. application Ser. No. (Attorney Docket No. 33475-US1, filed May 18, 2017). The disclosures of each of these applications are incorporated herein by reference in their entireties.

### FIELD OF THE DISCLOSURE

The present disclosure relates to methods and systems for associating information related to assay reagents, samples, and consumables.

### BACKGROUND

During the manufacture and use of biological reagents and consumables, products are typically coded and labeled for tracking purposes. Conventional systems use bar codes to identify reagents and consumables, with the bar codes applied to a carrier or vessel supporting the reagent and/or directly affixed to the consumable or to a container housing the consumable. Thereafter, the bar code is read by a bar code reader associated with a system used to conduct an experiment using that reagent or consumable. This enables the system to track the reagents and/or consumables presented to the system. Like bar codes, RFID technology can be used to track reagent or consumable usage. RFID technology offers several advantages to conventional bar code technology in that it does not require an optical path to read the information stored to the RFID and more data can be stored to an RFID. In this regard, reference is made to U.S. Pat. Nos. 7,187,286 and 8,770,471, and U.S. Patent Publication No. 2006/0199196, the disclosures of which are incorporated herein by reference.

Binding assays, e.g., immunoassays and nucleic acid hybridization assays, represent powerful tools to identify a very wide range of compounds. In general, detection is often limited when the analyte is present in small quantities because background signal caused by non-specifically bound materials interferes with the results. However, efforts to modify conventional assay techniques to improve sensitivity and specificity often yield more complex, labor intensive protocols that can be hampered by inefficiencies at each step, impacting the sensitivity and specificity of an assay. For example, in a complex assay requiring multiple binding events and/or reactions, if any one event or reaction is less than optimal, the sensitivity and specificity of the overall assay can suffer. There is a need for new techniques for improving assay performance by improving sensitivity and reducing non-specific binding.

### SUMMARY OF THE INVENTION

Improved detection and tracking methods are disclosed herein. One embodiment includes a method of detecting a target for a binding reaction between said target, a first binding reagent and a second binding reagent, wherein said first and second binding reagents are each capable of binding said target, the method comprising: (a) mixing a sample comprising said target and said first and second binding reagents, optionally in the presence of a conducting species, under conditions sufficient to form a complex comprising said target and said first and second binding reagents, wherein said first binding reagent is linked to a IC and said second binding reagent is linked to an antenna associated with said IC, such that upon complex formation said antenna is operatively connected with said IC, thereby transmitting a radio signal; and (b) detecting said target by measuring said radio signal transmitted by said IC upon complex formation.

Accordingly, the application provides a method of detecting a target for a binding reaction between said target, a first binding reagent and a second binding reagent, wherein said first and second binding reagents are each capable of binding said target, the method comprising: (a) mixing a sample comprising said target and said first and second binding reagents, optionally in the presence of a conducting species, under conditions sufficient to form a complex comprising said target and said first and second binding reagents, wherein said first binding reagent is linked to an RFID IC and said second binding reagent is linked to an RFID antenna associated with said IC, such that upon complex formation said antenna is operatively connected with said IC, thereby emitting a detectable signal; and (b) detecting said target by measuring said detectable signal emitted by said IC upon complex formation.

Also provided is a method of detecting a single stranded oligonucleotide target sequence via a hybridization reaction between said target, a first probe and a second probe, wherein said first probe comprises an RFID IC and said second probe comprises an antenna associated with said IC, the method comprising: (a) mixing a sample comprising said target and said first and second probes, optionally in the presence of a conducting species, under conditions sufficient to hybridize said first and second probes to said target, such that upon hybridization said antenna is operatively connected with said IC, thereby emitting a detectable signal; (b) detecting said target by measuring said detectable signal.

Another embodiment is a method of detecting a target for a binding reaction between said target, a first binding reagent and a second binding reagent, wherein said first and second binding reagents are each capable of binding said target, the method comprising: (a) mixing a sample comprising said target and said first and second binding reagents, optionally in the presence of a conducting species, under conditions sufficient to form a complex comprising said target and said first and second binding reagents, wherein said first binding reagent is linked to an RFID IC and said second binding reagent is linked to an antenna associated with said IC, such that upon complex formation said antenna is operatively connected with said IC, thereby emitting a detectable signal; (b) subjecting said complex to conditions sufficient to disrupt binding between one or more of said first binding reagent and said target, said second binding reagent and said target, or said first and second binding reagents and said target, thereby quenching said detectable signal; and (c) detecting said target by measuring a loss of said detectable signal.

Still further, the application provides a method of detecting a target oligonucleotide sequence for a binding reaction between said target oligonucleotide sequence, a forward primer sequence complementary to a 5′ region of said target sequence, and a reverse primer sequence complementary to a 3′ region of said target sequence, the method comprising(a) mixing a sample comprising said target and said forward and reverse primers under conditions sufficient to hybridize said forward and reverse primers to said target; (b) adding an RFID-modified Taqman probe to said complex under conditions sufficient to hybridize said probe to said target, wherein a first end of said probe comprises a IC and a second end of said probe comprises an antenna associated with said IC, such that upon hybridization of said probe to said target, optionally in the presence of a conducting species, said antenna is operatively connected with said IC, thereby emitting a detectable signal; (c) amplifying said target sequence; (d) displacing and cleaving said probe, thereby quenching said detectable signal; and (e) detecting said target by measuring a loss of said detectable signal.

One specific embodiment provided by the application is a modified Taqman probe comprising an RFID IC attached to the 5′ end of said probe and an antenna at the 3′ end of said probe. In addition, the application includes a modified Taqman probe comprising an RFID IC attached to the 3′ end of said probe and an antenna at the 5′ end of said probe.

Another embodiment includes a method of detecting a target for a binding reaction between said target, a first binding reagent and a second binding reagent, wherein said first and second binding reagents are each capable of binding said target, the method comprising: (a) mixing a sample comprising said target and said first and second binding reagents under conditions sufficient to form a complex comprising said target and said first and second binding reagents, wherein said first binding reagent is linked to an RFID IC and said second binding reagent is linked to an antenna associated with said IC, such that upon complex formation said antenna is operatively connected with said ag, thereby emitting a detectable signal; (b) adding a competitive reagent that competes for binding of the target with the second binding reagent, thereby forming a second complex comprising said target, said first binding reagent and said competitive reagent and quenching said detectable signal; and (c) detecting said target by measuring a loss of said detectable signal.

The application also provides a method of detecting a double stranded target oligonucleotide sequence via a binding reaction between a forward primer sequence complementary to a 5′ region of a first single stranded sequence of said target sequence, and a reverse primer sequence complementary to a 3′ region of a second single stranded sequence of said target sequence, wherein the forward primer sequence comprises an RFID IC and said reverse primer sequence comprises an antenna associated with said IC, the method comprising (a) denaturing said target sequence; (b) mixing said denatured target sequence and said forward and reverse primers under conditions sufficient to hybridize said forward and reverse primers to said first and second single stranded sequences, respectively; (c) subjecting hybrid sequences formed in step (b) to conditions sufficient to form a first primer extension product complementary to said first single stranded sequence and a second primer extension product complementary to said second single stranded sequence; (d) subjecting the products of step (c) to one or more cycles of polymerase chain reaction; (e) forming a complex comprising said first primer extension product hybridized to said second primer extension product, wherein upon complex formation said RFID IC is operably connected to said antenna thereby emitting a detectable signal; and (f) detecting said target by measuring said detectable signal.

A further embodiment includes a method of detecting a double stranded target oligonucleotide sequence for a binding reaction between said target oligonucleotide sequence, a forward primer sequence complementary to a 5′ region of a first single stranded sequence of said target sequence, and a reverse primer sequence complementary to a 3′ region of a second single stranded sequence of said target sequence, wherein the forward primer sequence comprises an antenna and said reverse primer sequence comprises an RFID IC associated with said antenna, the method comprising (a) denaturing said target sequence; (b) mixing said denatured target sequence and said forward and reverse primers under conditions sufficient to hybridize said forward and reverse primers to said first and second single stranded sequences, respectively; (c) subjecting hybrid sequences formed in step (b) to conditions sufficient to form a first primer extension product complementary to said first single stranded sequence and a second primer extension product complementary to said second single stranded sequence; (d) subjecting the products of step (c) to one or more cycles of polymerase chain reaction; (e) forming a complex comprising said first primer extension product hybridized to said second primer extension product, wherein upon complex formation said RFID IC is operably connected to said antenna thereby emitting a detectable signal; and (f) detecting said target by measuring said detectable signal.

Moreover, the application contemplates a method of detecting a single stranded oligonucleotide sequence comprising (a) mixing a sample comprising said sequence with a sensor comprising an RFID IC, an antenna associated with said IC, and a plurality of probes complementary to said sequence, (b) forming a complex including said sensor comprising said plurality of probes hybridized to said sequence, optionally in the presence of a conducting species, and thereby operatively connecting said RFID IC to said antenna to emit a detectable signal, and (c) detecting said sequence by measuring said detectable signal.

The application also contemplates a method of detecting a target comprising (a) mixing a sample comprising said target with a sensor comprising an RFID IC, an antenna associated with said IC, and a plurality of binding reagents capable of binding said target; (b) forming a complex including said sensor comprising said plurality of binding reagents bound to said target, optionally in the presence of a conducting species, and thereby operatively connecting said RFID IC to said antenna to emit a detectable signal; and (c) detecting said sequence by measuring said detectable signal.

Another embodiment provided by the application is a method of detecting a target comprising (a) contacting a sample comprising said target with a solid phase comprising (i) a plurality of RFID antennas, and (ii) a plurality of first binding reagents capable of binding said target, thereby forming a target-bound complex; (b) mixing said target-bound complex with a plurality of second binding reagents capable of binding said target, wherein each of said plurality of second binding reagents comprise an RFID IC associated with said antennas, optionally in the presence of a conducting species, thereby forming a detectable target-bound complex in which said RFID ICs are operatively connected with said antennas to emit a detectable signal; and (c) detecting said target by measuring said detectable signal.

Also provided is a method of detecting a target comprising contacting a sample comprising said target with a sensor comprising (i) an RFID antenna, (ii) an RFID IC associated with said RFID antenna, and (iii) a plurality of first binding reagents capable of binding said target, thereby forming a target-bound complex; mixing said target-bound complex with a plurality of second binding reagents capable of binding said target and thereby forming a detectable target-bound complex, wherein one or more second binding reagents are linked to a conducting species that operably connects the RFID antenna and IC upon formation of said detectable target-bound complex to produce a detectable signal; and detecting said target by measuring said detectable signal.

In addition, the application includes a sensor comprising a proximate end, a distal end, and a gate terminal spanning the proximate and distal end, an RFID antenna affixed to the proximate end and an RFID IC associated with the RFID antenna affixed to the distal end, and a plurality of first binding reagents capable of binding a target bound to the gate terminal. Finally, the application includes a kit comprising a sensor as described herein, and, in one or more separate containers, vials, or compartments, a plurality of second binding reagents capable of binding the target, wherein at least one of said plurality of second binding reagents is linked to a conducting species.

### BRIEF DESCRIPTION OF THE FIGURES

FIGS. 1A-1F are schematic illustrations of the method described herein. In FIG. 1A, a target is detected via the formation of a complex comprising the analyte and first and second binding reagents attached to an antenna (A) and IC, respectively. Upon formation of the complex, the antenna and IC are functionally connected to yield a detectable signal. In FIG. 1B, a specific embodiment of this method is illustrated in which the target is an antigen and the first and second binding reagents are antibodies that bind to different epitopes of the antigen. In FIG. 1C, the target is an oligonucleotide sequence and first and second primers bind to the sequence to form a detectable complex. FIGS. 1D-1F show specific embodiments in which a carbon nanotube is used as a conducting species to facilitate the functional connection between the antenna and IC.

FIGS. 2A-2C are schematic illustrations of alternative methods in which the loss of a detectable signal indicates the presence of the target. In FIG. 2A, the complex is initially formed to functionally connect the antenna and IC and thereby generate a detectable signal and then one or more bonds between the binding reagents and the target is/are disrupted to dissociate the complex, thereby reducing or eliminating the detectable signal. In FIG. 2B, the complex is an immunocomplex which is disrupted to reduce or eliminate the detectable signal. In FIG. 2C, the complex comprises a target oligonucleotide sequence bound to forward and reverse primers as well as a probe to which the antenna and IC are bound. The complex is initially detectable due to the proximity of the antenna and IC, but when the complex is disrupted in the presence of a nucleic acid polymerase having 5′ to 3′ nuclease activity, the polymerase releases a plurality of probe fragments and thereby disrupts the detectable signal.

FIGS. 3A-3B illustrate a further embodiment of an assay in which a target is detected via the loss of a detectable signal. In FIG. 3A, a complex is initially formed between a target and first and second binding reagents to which an antenna and IC are bound and functionally connected. When the complex forms, the antenna and IC are operably connected and a detectable signal is transmitted. The complex is then contacted with a competitive binding reagent that competes for binding of the binding reagent linked to the IC. When the competitor binds to the target, the IC-binding reagent is dislodged from the complex and the detectable signal is lost. Likewise, in FIG. 3B, the competitive reagent competes for binding with the antenna-binding reagent and when the competitor binds to the target, the antenna-binding reagent is dislodged from the complex and the detectable signal is lost.

FIG. 4A illustrates a specific embodiment in which a double stranded target is detected by subjecting the target to PCR using antenna- and IC-modified primers and the antenna and IC are ultimately incorporated into a complex between the extended primers. FIG. 4B represents an embodiment in which the IC and antenna are incorporated into a sensor. When the target nucleic acid binds to the sensor, the circuit is completed such that the antenna and IC are operatively connected to transmit a detectable signal. In a specific embodiment, the sensor includes a plurality of primers or probes complementary to at least a portion of the target sequence such that a plurality of target molecules bind to the sensor. FIG. 4C shows yet another embodiment in which the IC and antenna are incorporated into a sensor in which antibodies are bound to the sensor and the circuit is completed when the analyte is bound to the bound antibodies. FIGS. 4D-4F illustrate additional embodiments of the assays depicted in FIGS. 4A-4C in which a conducting species comprising a carbon nanotube is employed to facilitate the operable connection between the antenna and IC. In FIG. 4D, the conducting species, e.g., carbon nanotube, is linked to a DNA intercalator or DNA binding protein that recognizes and binds to the duplex formed in the final step of the assay. In the embodiments depicted in FIGS. 4B-4C and 4E-4F, the solid phase is a sensor.

FIGS. 5A-5D show specific embodiments of an assay in which a protein is the target. A plurality of binding reagents are bound to solid phase (in FIGS. 5A-5B the solid phase is a particle and in FIGS. 5C-5D the solid phase is a lateral support, e.g., an assay container, slide, chip, flow cell, cartridge or plate). The solid phase also includes a plurality of antennas. The target molecule binds to the binding reagents and then a second binding reagent is added, wherein the second binding reagent is bound to a IC. Once the complex is fully formed, the antenna is operably connected to the IC and a detectable signal is transmitted. In an alternative embodiment, the solid phase includes a plurality of ICs and the second binding reagents are each linked to an antenna. In FIGS. 5B and 5D, a conducting species (“CS”), e.g., a carbon nanotube, is employed to facilitate the operable connection between the antenna and IC.

FIGS. 6A-6D show an alternative embodiment of the assay illustrated in FIGS. 5A-5D wherein the target is an oligonucleotide. As in FIGS. 5A-5D, in FIGS. 6A and 6C, the solid phase is a particle and in FIGS. 6B and 6D, the solid phase is a lateral support. The solid phase is linked to a plurality of probes complementary to a target as well as a plurality of antennas. The probes hybridize to the target, and then a plurality of secondary probes each linked to a IC is added, forming a complex in which the antenna is operably connected to the IC and a detectable signal is transmitted. Alternatively, the solid phase includes a plurality of ICs and the secondary probes are each linked to an antenna. In FIGS. 6B and 6D, conducting species (“CS”) are employed to facilitate the operable connection between the antenna and IC.

FIGS. 7A-7B illustrate the configuration of a sensor (FIG. 7A) and additional components that can be included in a kit supplied with a sensor (FIG. 7B).

### DETAILED DESCRIPTION OF THE INVENTION

Unless otherwise defined herein, scientific and technical terms used in connection with the present disclosure shall have the meanings that are commonly understood by those of ordinary skill in the art. Further, unless otherwise required by context, singular terms shall include pluralities and plural terms shall include the singular. The articles “a” and “an” are used herein to refer to one or to more than one (i.e., to at least one) of the grammatical object of the article. By way of example, “an element” means one element or more than one element.

The methods and systems described herein are improvements to conventional assay methods that combine the selectivity of proximity binding assays and component tracking methods provided by proximity-based information storage/tracking systems, including but not limited to RFID and/or microtransponder systems. Briefly, the assay methods described herein enable the detection of a target by monitoring the formation of a complex between the target and a pair of binding reagents each linked to a component of a detection/tracking system. In the absence of the target, the detection/tracking components on the pair of binding reagents are not operably connected because they are not in sufficient proximity to one another to interact and form a complete circuit between the RFID antenna and IC. However, if the target is present, the complex forms and the detection/tracking components are brought into sufficient proximity to operably connect the components and thereby transmit a detectable signal. The method optionally includes the addition of a conducting species to facilitate the operable connection between antenna and IC in the system. In addition, because the binding reagents are labeled by detection/tracking components comprising stored information about the assay components, the detection system also functions as a component tracking system. Therefore, the detection/tracking components perform a dual purpose: (i) providing a detectable signal in the presence of a target molecule; and (ii) carrying information about the assay components.

The methods described herein are useful in and can include any type of diagnostic or analytical method known in the art. Such analytical methods include but are not limited to clinical chemistry assays (e.g., measurements of pH, ions, gases and metabolites), hematological measurements, nucleic acid amplification assays (e.g., polymerase chain reaction (PCR), ligase chain reaction assays, strand displacement amplification, self-sustained synthetic reaction, isothermal amplification, e.g., helicase-dependent amplification and rolling-circle amplification), immunoassays (e.g., direct, sandwich and/or competitive immunoassays and serological assays), oligonucleotide ligation assays, nucleic acid sequencing processes, and nucleic acid hybridization assays. In a specific embodiment, the analytical method is a nucleic acid amplification assay, e.g., PCR or ligase chain reaction. Alternatively, the method is an immunoassay, e.g., a direct, sandwich, or competitive immunoassay. The immunoassay can be a serological assay. The corresponding system can include a reaction module configured to perform the selected diagnostic assay, as well as memory, a processor, and a display. The reaction module includes one or more sample processing modules and each sample processing module comprises one or more units or stations for carrying out the various steps required to process a sample. If the assay is a nucleic acid amplification assay, the sample processing module can include a reaction chamber and a thermoelectric cooling device, e.g., a thermal cycler, and optionally one or more of the following: a sample dispensing station, a separation station, and one or more consumable and/or reagent storage stations. The reaction chamber is configured to house a sample during one or more nucleic acid amplification reaction steps. In addition, the nucleic acid amplification module also includes at least one control unit electrically connected to one or more of the sample processing modules. The control unit also includes an analysis module configured to analyze a nucleic acid to obtain a detectable signal.

Memory can include any combination of any type of volatile or non-volatile memory, such as random-access memories (RAMs), read-only memories such as an Electrically-Erasable Programmable Read-Only Memory (EEPROM), flash memories, hard drives, solid state drives, optical discs, and the like. Memory can be a single device or it can also be distributed across two or more devices. A processor can include one or more processors of any type, such as central processing units (CPUs), graphics processing units (GPUs), special-purpose signal or image processors, field-programmable gate arrays (FPGAs), tensor processing units (TPUs), and so forth. A processor can be a single device or distributed across any number of devices. The display can be implemented using any suitable technology, such as LCD, LED, OLED, TFT, Plasma, etc. In some implementations, the display may be a touch-sensitive display (a touchscreen).

The system can also be operably connected to one or more computing devices (not shown) such as desktop computers, laptop computers, tablets, smartphones, servers, application-specific computing devices, or any other type(s) of electronic device(s) capable of performing the techniques and operations described herein. In some embodiments, the elements of the system and the subcomponents of each element can be provided in a single device or as a combination of two or more devices together achieving the various functionalities discussed herein. For example, a nucleic acid amplification module may include one or more server computers and one or more client computers communicatively coupled to each other via one or more local-area networks and/or wide-area networks. Finally, the system can also include one or more peripheral devices (e.g., a printer and keyboard), and the computer subsystems can be interconnected via a system bus. Peripherals and input/output (I/O) devices, which couple to an I/O controller, can be connected to the system by any means known in the art, such as a serial port. For example, a serial port or external interface (e.g. Ethernet, Wi-Fi, etc.) can be used to connect the system to a wide area network such as the Internet, a mouse input device, or a scanner. The interconnection via system bus allows the central processor to communicate with each subsystem and to control the execution of instructions from system memory or the storage device(s), as well as the exchange of information between subsystems. The system memory and/or the storage device(s) may embody a computer readable medium.

A computer system can include a plurality of the same components or subsystems, e.g., connected together by external interface or by an internal interface. In some embodiments, computer systems, subsystem, or apparatuses can communicate over a network. In such instances, one computer can be considered a client and another computer a server, where each can be part of a same computer system. A client and a server can each include multiple systems, subsystems, or components.

It should be understood that any of the embodiments of the present disclosure can be implemented in the form of control logic using hardware (e.g. an application specific integrated circuit or field programmable gate array) and/or using computer software with a generally programmable processor in a modular or integrated manner. As used herein, a processor includes a multi-core processor on an same integrated chip, or multiple processing units on a single circuit board or networked. Based on the disclosure and teachings provided herein, a person of ordinary skill in the art will know and appreciate other ways and/or methods to implement embodiments of the present disclosure using hardware and a combination of hardware and software.

Any of the software components or functions described in this application may be implemented as software code to be executed by a processor using any suitable computer language such as, for example, Java, C++ or Perl using, for example, conventional or object-oriented techniques. The software code may be stored as a series of instructions or commands on a computer readable medium for storage and/or transmission, suitable media include random access memory (RAM), a read only memory (ROM), a magnetic medium such as a hard-drive or a floppy disk, or an optical medium such as a compact disk (CD) or DVD (digital versatile disk), flash memory, and the like. The computer readable medium may be any combination of such storage or transmission devices.

Such programs may also be encoded and transmitted using carrier signals adapted for transmission via wired, optical, and/or wireless networks conforming to a variety of protocols, including the Internet. As such, a computer readable medium according to an embodiment of the present disclosure may be created using a data signal encoded with such programs. Computer readable media encoded with the program code may be packaged with a compatible device or provided separately from other devices (e.g., via Internet download). Any such computer readable medium may reside on or within a single computer program product (e.g. a hard drive, a CD, or an entire computer system), and may be present on or within different computer program products within a system or network. A computer system may include a monitor, printer, or other suitable display for providing any of the results mentioned herein to a user.

Any of the methods described herein may be totally or partially performed with a computer system including one or more processors, which can be configured to perform the steps. Thus, embodiments can be directed to computer systems configured to perform the steps of any of the methods described herein, potentially with different components performing respective steps or a respective group of steps. Although presented as numbered steps, steps of methods herein can be performed at a same time or in a different order. Additionally, portions of these steps may be used with portions of other steps from other methods. Also, all or portions of a step may be optional. Additionally, any of the steps of any of the methods can be performed with modules, circuits, or other means for performing these steps.

“Binding reagent” includes but is not limited to, any molecule known in the art to be capable of, or putatively capable of, binding an analyte of interest. An analyte of interest may include but is not limited to, e.g., a whole cell, a subcellular particle, virus, prion, viroid, nucleic acid, protein, antigen, lipoprotein, lipopolysaccharide, lipid, glycoprotein, carbohydrate moiety, cellulose derivative, antibody or fragment thereof, peptide, hormone, pharmacological agent, cell or cellular components, organic compounds, non-biological polymer, synthetic organic molecule, organo-metallic compounds or an inorganic molecule present in the sample. Thus, the binding reagents include but are not limited to receptors, ligands for receptors, antibodies or binding portions thereof (e.g., Fab), proteins or fragments thereof, nucleic acids, oligonucleotides, primers, probes, glycoproteins, polysaccharides, antigens, epitopes, cells and cellular components, subcellular particles, carbohydrate moieties, enzymes, enzyme substrates, lectins, protein A, protein G, organic compounds, organometallic compounds, viruses, prions, viroids, lipids, fatty acids, lipopolysaccharides, peptides, cellular metabolites, hormones, pharmacological agents, tranquilizers, barbiturates, alkaloids, steroids, vitamins, amino acids, sugars, nonbiological polymers, biotin, avidin, streptavidin, organic linking compounds such as polymer resins, lipoproteins, cytokines, lymphokines, hormones, synthetic polymers, organic and inorganic molecules, etc. Nucleic acids and oligonucleotides can refer to DNA, RNA and/or oligonucleotide analogues including but not limited to: oligonucleotides containing modified bases or modified sugars, oligonucleotides containing backbone chemistries other than phosphodiester linkages, and/or oligonucleotides, that have been synthesized or modified to present chemical groups that can be used to form attachments to (covalent or non-covalent) to other molecules.

For example, the binding reaction is a nucleic acid binding reaction wherein the target is an oligonucleotide and the first and second binding reagents are primer sequences complementary to the 5′ and 3′ ends of the target oligonucleotide sequence, respectively. Alternatively, if the target is a protein antigen, the first and second binding reagents are antibodies that bind to different epitopes of the antigen. The skilled artisan will readily appreciate the wide array of binding reactions that can be exploited using this technique without departing from the spirit or scope of the invention, including, but not limited to, ligand-receptor, enzyme-substrate, antibody-antigen, nucleic acid-complement, serology, etc. Likewise, the skilled artisan in the field of binding assays will readily appreciate the scope of binding reagents and companion binding partners that may be used in the present methods. A non-limiting list of such pairs include (in either order) receptor/ligand pairs, antibodies/antigens, natural or synthetic receptor/ligand pairs, hapten/antibody pairs, antigen/antibody pairs, epitope/antibody pairs, mimitope/antibody pairs, aptamer/target molecule pairs, hybridization partners, oligonucleotide/DNA-binding protein, and intercalater/target molecule pairs. In one embodiment, the binding assays employ antibodies or other receptor proteins as capture and/or detection reagents for an analyte of interest. The term “antibody” includes intact antibody molecules (including hybrid antibodies assembled by in vitro re-association of antibody subunits), antibody fragments and recombinant protein constructs comprising an antigen binding domain of an antibody (as described, e.g., in Porter, R. R. and Weir, R. C. J. Cell Physiol., 67 (Suppl); 51-64 (1966) and Hochman, 1. Inbar, D. and Givol, D. Biochemistry 12: 1130 (1973)), as well as antibody constructs that have been chemically modified.

As used herein, “reagent” includes but is not limited to, any biological reagent that might be used in an analytical method, e.g., solutions comprising one or more of the following: detergent, buffer, diluent, calibrators, controls, co-reactants, enzymes, water, inorganic or organic solvents, nucleic acids, nucleotides (dNTPs and ddNTPs), oligonucleotides, DNA, RNA, PNA, primers, probes, adapters, aptamers, antibodies or fragments thereof, antigens, small molecules, e.g., drugs or prodrugs, streptavidin, avidin, and biotin, and mixtures thereof. Generally, “reagent” includes any substance apart from a biological sample that is used in the preparation for and/or conduct of an assay, including but not limited to, a nucleic acid amplification assay (e.g., PCR), a nucleic acid sequencing process, an immunoassay, a cellular assay, etc. In a specific embodiment, “reagent” includes a reagent used in a nucleic acid amplification reaction, e.g., PCR Master Mix and reagents required for isothermal amplification, including but not limited to, DNA polymerase, e.g., Taq polymerase, dNTPs, MgCl₂, buffers, helicase, nicking enzyme, or mixtures thereof. In another embodiment, “reagent” includes a reagent used in a sequencing process and/or library preparation process, including but not limited to, sequencing adapters, controls, primers, DNA polymerase, dNTPs, labeled ddNTPs, molecular tags, expression vector(s), template, ligase master mix, etc. An additional embodiment is a cellular assay, e.g., an assay described in U.S. Pat. No. 9,481,903, which is incorporated herein by reference, wherein a “reagent” can include but is not limited to, a population of engineered transduction particles, a biologic or abiologic vector, bacterial nutrient media, buffers, surfactant, or other components to facilitate cell growth. A further embodiment is a “reagent” used in an immunoassay, e.g., antibodies or fragments thereof, antigens, bovine serum albumin, streptavidin, avidin, biotin, labeled assay components, e.g., components including a radiolabel, chemiluminescent label, electrochemiluminescent, or luminescent label, fluorophore, etc., immunoassay coreactants, e.g., tertiary amines (if the assay is an electrochemiluminescent assay, a coreactant including tripropyl amine is used in the assay), etc. Additionally, a reagent in an assay can comprise an identifier conjugated to the reagent via a non-reactive substance inert to the conditions of the assay protocol.

“Sample” refers to any emulsion, suspension, or liquid sample matrix that can be analyzed in the assay systems described above. As used herein, “sample” includes, but is not limited to samples containing or derived from, for example, cells (live or dead) and cell-derived products, immortalized cells, cell fragments, cell fractions, cell lysates, organelles, cell membranes, hybridoma, cell culture supernatants (including supernatants from antibody producing organisms such as hybridomas), waste or drinking water, food, beverages, pharmaceutical compositions, blood, serum, plasma, hair, sweat, urine, feces, tissue, biopsies, effluent, separated and/or fractionated samples, separated and/or fractionated liquids, organs, saliva, animal parts, animal byproducts, plants, plant parts, plant byproducts, soil, minerals, mineral deposits, water, water supply, water sources, filtered residue from fluids (gas and liquid), swipes, absorbent materials, gels, cytoskeleton, protein complexes, unfractionated samples, unfractionated cell lysates, endocrine factors, paracrine factors, autocrine factors, cytokines, hormones, cell signaling factors and or components, second messenger signaling factors and/or components, cell nucleus/nuclei, nuclear fractions, chemicals, chemical solutions, structural biological components, skeletal (ligaments, tendons) components, separated and/or fractionated skeletal components, hair, fur, feathers, hair fractions and/or separations, skin, skin samples, skin fractions, dermis, endodermis, eukaryotic cells, prokaryotic cells, fungus, yeast, antibodies, antibody fragments, immunological factors, immunological cells, drugs, therapeutic drugs, oils, extracts, mucous, fur, oils, sewage, environmental samples, organic solvents or air. The sample may further comprise, for example, water, organic solvents or mixtures thereof. The sample can also include nucleic acid (e.g., DNA or RNA) that has been isolated from a biological material. The sample can be purified, in whole or in part. The samples contemplated herein can be fresh, refrigerated, frozen, reconstituted, and/or combined with one or more preservatives, stabilizers, or additives.

“Component” is referred to herein as any reagent, sample, or consumable that can be used in an assay system. Certain types of information stored to an identifier is referred to herein as “component information” because that information can relate to a reagent, sample or consumable and it is not distinguished by the type of component.

An “identifier” is a storage medium comprising memory to store information related to the sample, reagent, and/or consumable, e.g., its history and/or its use. In a specific embodiment, the identifier is an RFID, i.e., radio frequency identification. As described in more detail below, using RFID, the electromagnetic or electrostatic coupling in the RF portion of the electromagnetic spectrum is used to transmit signals.

Detection/Tracking Systems & Components

The methods described herein employ identifier technology as a detection and tracking mechanism. One example of an identifier system is an RFID, i.e., radio frequency identification device, which includes a storage medium comprising memory to store information related to the sample, reagent, and/or consumable, e.g., its history and/or its use, and the electromagnetic or electrostatic coupling in the RF portion of the electromagnetic spectrum is used to transmit a detectable signal.

RFIDs can be classified as active or passive. Active RFID systems have three essential components: (a) a reader, transceiver or interrogator, (b) antenna, and (c) a transponder or IC programmed with information. Active RFID tags possess a microchip circuit (transponder or IC) and an internal power source, e.g., a battery, and when operably connected to an antenna, the active RFID tag transmits a signal from the microchip circuit through the power obtained from the internal battery. In general, two different types of active RFID tags are commercially available: transponders and beacons. In a system that uses an active transponder, the reader sends a signal and when the antenna and tag are operably connected, the tag will send a signal back with the relevant information programmed to the transponder. In a system that uses an active beacon, the beacon will send out a signal on a periodic basis and it does not rely on the reader's signal. Therefore, if an active RFID system is used in the methods described herein, one binding reagent includes an antenna and the other binding reagent includes a tag, and upon complex formation the antenna and tag are brought into proximity to be operably connected and capable of relaying a detectable signal to a reader with information programmed to the tag. The operable connection can be facilitated using a conducting species, as described herein below.

Passive systems also comprise (a) a reader, transceiver or interrogator, (b) antenna, and (c) a tag programmed with information. A passive RFID tag includes a microchip or integrated circuit (IC), and it may contain the antenna as an integral component of the tag or as a separate device, but in a passive RFID system the tag does not include a power source. In one configuration of a passive system, the antenna can be an internal component of the tag, i.e., the antenna and IC can be contained in a single device instead of segregated into separate devices, but until operably connected in the device, the antenna and IC do not interact. Alternatively, the antenna and IC can be provided on separate components as described above regarding the active RFID systems. As the name implies, passive systems wait for a signal from an RFID reader which sends energy to the antenna which converts that energy into an RF wave that is sent into the read zone. Once the tag is read within the read zone, the RFID antenna draws in energy from the RF waves. The energy moves from the tag's antenna to the IC and powers the chip which generates a signal that is sent back to the RF system. The change in the electromagnetic or RF wave is detected by the reader and this constitutes the detectable signal transmitted by the RFID tag. A conventional passive RFID tag consists of an IC and internal antenna and this basic structure is commonly referred to as an RFID inlay. Countless other types of passive RFID tags exist on the market, but all tags generally fall into two categories—inlays or hard tags. Hard RFID tags are durable and made of plastic, metal, ceramic and even rubber. They come in all shapes and sizes and are typically designed for a unique function, material, or application.

Passive RFID tags do not all operate at the same frequency. There are several frequencies within which passive RFID tags operate. The frequency range, along with other factors, strongly determines the read range, attachment materials, and application options.

125-134 KHz—Low Frequency (LF)

5-7 MHz—High Frequency (HF)

13.56 MHz—HF & Near-Field Communication (NFC)

433 MHz—Ultra-High Frequency (UHF)

865-960 MHz—UHF

2.4 GHz—UHF

5.2-5.8 GHz—UHF

In a specific embodiment in which a passive RFID system is used, an UHF frequency is used, e.g., of between 1.0-3.0 GHz, particularly, 1.5, 2.0, and/or 2.45 GHz.

In the passive systems used in the methods described herein, the passive tag includes the IC and the antenna is provided in the same or a separate component but the IC and antenna are not operably connected until target is effectively bound to form a complete circuit between the antenna and IC. In the absence of a target, the antenna and IC cannot be operably connected and therefore, even in the presence of energy transmitted by a reader, the tag cannot generate a signal. However, when the target is present and bound to the binding reagent(s), optionally in the presence of a conducting species, the antenna and IC are operably connected; the antenna converts energy transmitted by the reader into an RF wave that is sent into the read zone. Once the tag is read within the read zone, the antenna draws in energy from the RF waves. The energy moves from the antenna to the IC and powers the chip which generates a signal back to the RF system. The change in the electromagnetic or RF wave is detected by the reader and this constitutes the detectable signal transmitted by the RFID tag.

In the present disclosure, the term “operably connected” or “operable connection” refers to the formation of a complete electrical circuit between the antenna and IC such that energy moves from the antenna to the IC and powers the chip which generates a signal back to the RF system. The operable connection between an antenna and IC can be facilitated by the presence of a conducting species, as described below, wherein the conducting species is positioned in the system to act as a bridge between the antenna and IC.

Active and/or passive RFID systems are available from Motorola, Alien Technology, Applied Wireless RFID, CAEN RFID, GAO RFID, Impinj, Mojix, NXP Semiconductors, ThingMagic, Avery Dennison, Invengo, Omni-ID, Confidex, Metalcraft, and Smartrac Technology. In a specific embodiment, the RFID system is a system such as that provided by Philtech, Inc. (Tokyo, Japan) or Hitachi Ltd. (Japan). The Philtech system is described, e.g., in Mura et al., “RF-Powder: Fabrication of 0.15-mm Si-powder Resonating at Microwave Frequencies” Proceedings of the 37^{th} European Microwave Conference, October 2007, pp. 392-395, as well as U.S. Application/Patent Nos. 20080198000; U.S. Pat. Nos. 7,777,631; 7,839,276; 7,997,495; and the Hitachi system is described in U.S. Application/Patent Nos. 20060077062; U.S. Pat. No. 7,378,971; and Nozawa, “Hitachi Achieves 0.05-mm Square Super Micro RFID Tag, ‘Further Size Reductions in Mind’” Nikkei Technology, Tech & Industry Analysis from Japan/Asia online, Feb. 20, 2007, the disclosures of which are incorporated herein by reference.

For example, U.S. Pat. No. 8,766,802 to Philtech, Inc. the disclosure of which is incorporated herein by reference, relates to a base data management system that includes a base data reader including reading means that reads specific data of particles fixed to a base and transmitting means that transmits the specific data read by the reading means and reader information. The system also includes a computer including data receiving means that receives the specific data and reader information transmitted from the base data reader through a network, storage means that stores the specific data and reader information received by the data receiving means, and output means that processes the data stored in the storage means according to the application and outputs the processed data. The system includes a base data reader that reads specific data of a base and transmits the specific data and reader information, and a computer that receives and stores the specific data and reader information transmitted from the base data reader through a network, and outputs the data and information as required.

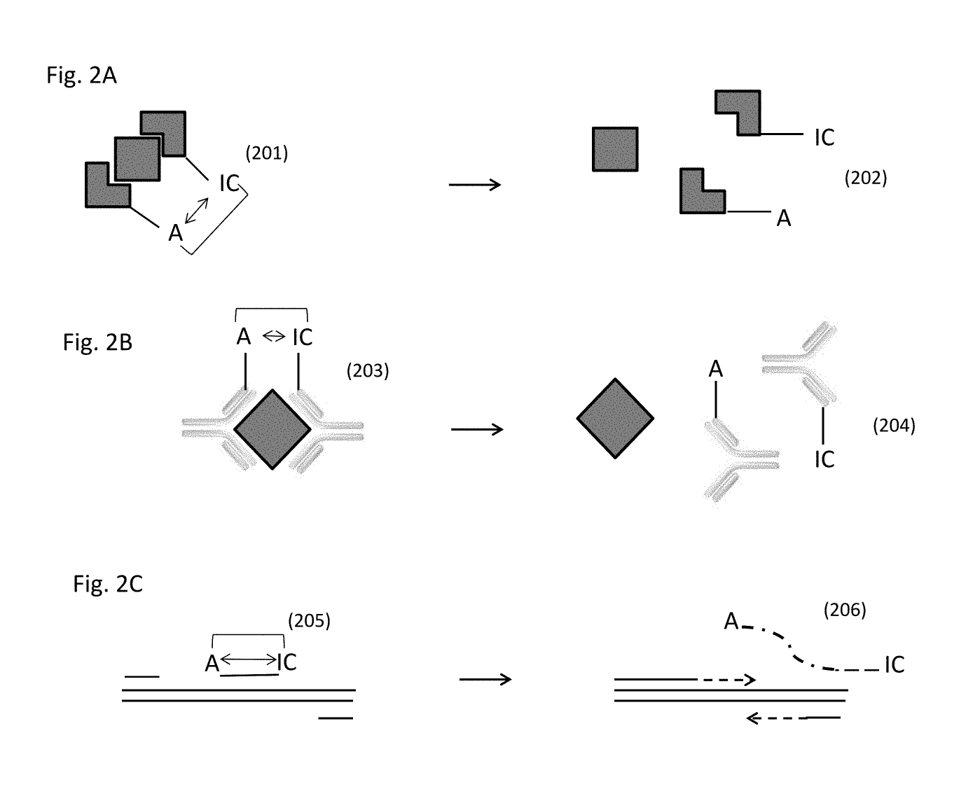

U.S. Pat. No. 8,766,802 describes a base used in a base data management system is described (see, e.g., FIG. 2 of U.S. Pat. No. 8,766,802, the disclosure of which is incorporated herein by reference in its entirety). The base depicted in the figure includes an RF powder. In the embodiment shown, a single type of a large number of RF powder particles are disposed on a surface of a base by printing or the like. The RF powder particles respond to a high frequency electromagnetic field having a single frequency. The “RF powder” refers to a powder comprised of a large number of particles, each having an electrical circuit element that transmits and receives signals to or from external readers by radio (in a high frequency electromagnetic field). The particles are generally treated as a powder collectively.

In the present context, a quantity of RF powder particle components, i.e., an RF powder antenna and IC, respectively, are bound to a surface, e.g., a sensor, that also comprises a plurality of binding reagents for a target of interest bound to the surface of the sensor. In a specific embodiment, the antenna and IC are separated in the sensor by an insulating layer and upon formation of a binding complex between a target and binding member on the surface of the sensor, the antenna and IC are operatively connected to yield a detectable RFID signal. The sensor is mixed with sample to form a target-bound complex on the surface of the sensor, and one or more second binding reagents for the target are mixed with the sensor, and optionally with a conducting species, to form an operable connection between the RF antenna and IC, thereby yielding a detectable signal. Each sensor can be configured to produce a unique resonant frequency and therefore, in an additional embodiment, a plurality of sensors can be employed to interrogate a complex sample having a plurality of target species in a multiplexed assay. Each sensor in the plurality includes a set of individually detectable RF antennas/ICs that, when suitably admixed with a target of interest, is individually detectable. Thus, using this method, a multiplexed detection system is also provided.

Reference is also made to U.S. Pat. No. 8,318,047 to Philtech, Inc., the disclosure of which is incorporated herein by reference in its entirety, which discloses an RF powder-containing liquid, i.e., water, alcohol, or ink, which contains a large amount of RF powder particles mixed with a pigment to distinguish the characteristic frequency of the RF powder suspended in the liquid from another liquid having a different frequency and pigment. Reference is further made to U.S. Pat. No. 8,154,456 to Philtech, Inc., the disclosure of which is incorporated herein by reference in its entirety.

In the RF powder particle, an insulating layer (SiO2 or the like) is formed on, for example, a silicon (Si) substrate, and a plural-turn coil (inductance element) and a capacitor (capacitance element) are formed on the insulating layer by a film-forming technique. The coil and the capacitor formed on the insulating layer are coupled with a high frequency magnetic field having a specific frequency (for example, 2.45 GHz) and resonate. The number of turns and the length of the coil are arbitrarily set to obtain an intended resonance frequency. The shape of the coil may also be changed. The pad electrodes of the capacitor, and the dielectric material disposed between the pad electrodes and its thickness can also be appropriately designed according to an intended frequency. Moreover, the RF powder particle responds to only a high frequency electromagnetic field depending on the resonance frequency of the tank circuit. Thus, the RF power particle functions as a “powder circuit element” that is coupled with a magnetic field of a designed frequency to resonate.

The substrate of a base of the RF powder particle is made of silicon, and is provided with the insulating layer over the surface thereof. As an alternative to the silicon substrate, a substrate made of a dielectric (insulative) material, such as glass, a resin, or a plastic, may be used. If a glass substrate or the like is used, the insulating layer is not necessary because the material of such a substrate is intrinsically insulative (dielectric).

In a specific embodiment, the RFID system comprises an antenna tuned to a unique resonant frequency, such that each reagent and sample in the system are each tuned to a unique resonant frequency distinguishable from the frequencies of other components in the system. In this regard, the antenna can comprise carbon single-walled nanotubes and the unique resonant frequency of the antenna is adjustable by modifying the length of the nanotubes.

The RFID reader/writer has a read terminal and reads information provided from the RF powder particles using radio-frequency electromagnetic waves (RF) in a specific frequency band, e.g., ranging from about 1.0-3.0 GHz, e.g., 1.5-2.5 GHz. The frequencies used in each of the plurality of RF powder particles can be different from each other, for example, one set of particles can use 1.9 GHz, a second set uses 2.0 GHz, and a third set uses 2.45 GHz. Hence, the RFID reader/writer is configured to read the electromagnetic waves of, for example, 1.5 to 3.0 GHz frequency band. In order to read information from each of the plurality of RF powder particles via the read terminal, the reader/writer performs a scanning operation in a certain direction along the outside of the vessel or container, and also changes the frequency used for transmission/reception within the specific frequency band. Only those particles that use the specific frequency band being scanned will generate a detectable signal, i.e., respond to the electromagnetic wave at the specific frequency band. Therefore, if there are three different sets of particles in a vessel, the first using 1.9 GHz, the second using 2.0 GHz, and the third using 2.45 GHz, when the read/writer performs a scanning operation at 2.0 GHz, only the second set of particles will respond to the read/writer, but the first and third sets of particles will not.

In an alternative or additional embodiment, the identifier system includes a microtransponder tag, e.g., a p-Chip® (available from Pharma Seq, Inc., Monmouth Junction, N.J.), which are ultra-small identifiers that carry a unique serial number. These identifiers are approximately 500×500 microns and nominally 100 microns thick. Unlike RFID technology, instead of using radio frequency detection, microtransponder tags include photocells that, when illuminated by light from a reader, provide power and synchronization signals for the tag's electronic circuits. Additionally, each tag includes an on-chip antenna that transmits its unique serial number when stimulated by pulsed, laser light. Therefore, as applied to the methods described herein if a microtransponder tag is used the system operates much like a passive RFID system, in that in the absence of target, the antenna and IC are not operably connected but upon complex formation, the IC is operably connected to the antenna and powered to transmit a unique serial number upon stimulation. Therefore, in this embodiment, the detectable signal is the transmission of information, e.g., a unique serial number. In this embodiment, the reader can identify the tag via the unique serial number and query a storage medium on the system or network for additional component information associated with that serial number, e.g., the assay protocol script and the associated requirements list.

The methods and systems described herein can be used for detection target species, but additional systems are optionally used as supplemental tracking methods. For example, RFID and/or microtransponder systems can be supplemented by one or more additional identifiers, e.g., bar codes, RFID, EPROM, EEPROM, ICC, flash memory, or combinations thereof. For example, reagents or samples can include suspended RFID identifiers and one or more containers, vessels, or compartments used in the system, e.g., in the preparation for and/or conduct of an assay in the system can be labeled with a supplemental identifier, e.g., one or more RFID, bar codes, EPROM, EEPROM, ICC, flash memory, or combinations thereof. In this embodiment, the system is operably connected to a plurality of readers each configured to read information from a distinct type of identifier. Certain information can be stored on one identifier and other information on an additional identifier of the same or different type. For example, a reagent and/or sample can include suspended RFIDs that include reagent and sample information, respectively, e.g., the type of reagent and/or sample, and for a sample, patient identification information, whereas the container housing the reagent or sample can include another identifier, e.g., a bar code or other type of non-volatile memory, used to store additional information. For example, if the container houses a reagent, a bar code can be included on the container with reagent information comprising manufacturer information or lot specific parameters for that reagent. In this regard, reference is made to U.S. Provisional Application Ser. No. 62/338,620, filed May 19, 2016, and U.S. application Ser. No. [attorney docket no. 33475-US1, filed May 18, 2017], the disclosure of which is incorporated herein by reference.

The reader controls the operation of the detection/tracking system components and other components of the assay system. For example, the reader optionally includes or is operably connected to a micro-controller to interface with the RFID/microtransponder non-volatile memory over a communication interface, which can incorporate conventional interface architectures and protocols such as I^{2C}, a two line serial bus protocol. The microcontroller addresses the non-volatile memory and performs write, read and erase operations on the memory.

Sensors for biological detection are well known in the art. See, e.g., Veigas et al., “Field Effect Sensors for Nucleic Acid Detection: Recent Advances and Future Perspectives,” Sensors (2015): 10380-10398. For example, the field effect transistor (FET) is an approach to electrical DNA detection and characterization having small dimensions, fast response, integration into arrays and the possibility of low-cost mass production. The simultaneous analysis of various DNA/RNA targets in miniaturized analytical systems has allowed for the development of comprehensive assay platforms, e.g., as employed in the IonTorrent System (Thermo Fisher Scientific, Waltham, Mass.). Field effect sensors can include either a metal insulator-semiconductor capacitor (MIS), or a metal-oxide semiconductor field effect transistor (MOSFET). Field effect sensors may be described as three electrode devices where the current flow between the source and drain electrodes can be modulated by varying the potential applied to the gate and source electrodes. A source terminal is a terminal through which the carriers enter the channel, a drain terminal is a terminal through which the carriers leave the channel, and a gate terminal is a terminal that modulates the channel conductivity. This gate permits electrons to flow through or blocks their passage by creating or eliminating a channel between the source and drain. The semiconductor layer is separated from the gate electrode by an insulator layer that prevents current flow between them. The current-control mechanism is based on an electric field generated by the voltage applied to the gate. A field effect sensor can be configured as a biosensor by modifying the gate terminal with binding reagents specific for the analyte of interest. The binding of a biomolecule results in depletion or accumulation of carriers caused by a change of electric charges on the gate terminal.

In the specific example of a sensor for the detection of a nucleic acid target sequence, a hybridization event is used to detect the target sequence. Detection is usually achieved by DNA modified sensors via immobilizing probes onto the sensor's surface. Because DNA is an intrinsically charged molecule due to the phosphate backbone, the charge density increases near the sensor's surface, yielding a response. This effect can be enhanced in the presence of a conducting species, as described below. Hence, provided herein is a sensor comprising a proximate end, a distal end, and a gate terminal spanning the proximate and distal end. An RFID antenna is affixed to the proximate end and an RFID IC associated with the RFID antenna is affixed to the distal end, and a plurality of first binding reagents capable of binding a target are bound to the gate terminal. Also provided is a kit including the sensor described herein, and in one or more separate containers, vials, or compartments, a plurality of second binding reagents capable of binding the target, wherein at least one of said plurality of second binding reagents is linked to a conducting species.

A conducting species may be used to facilitate the operable connection between the RF antenna and IC. For example, the conducting species comprises carbon fibrils, carbon nanotubes, graphitic nanotubes, graphitic fibrils, carbon tubules, fibrils, buckeytubes, and/or buckyballs (buckeytubes and buckyballs are forms of buckminsterfullerene (C60)). Alternatively, the conducting species can include ferrocene and/or ferrocene derivatives. In a particular embodiment, the conducting species is a carbon nanotube. Biosensors employing carbon nanotubes are known. See, e.g., Yang et al., “Carbon nanotube based biosensors,” Sensors and Actuators B 207 (2015): 690-715. Carbon nanotubes are seamless hollow tubes composed of rolling graphite sheets. Single- and multi-walled carbon nanotubes are known. In general, single-walled carbon nanotubes (SWCNT) are single molecular nanomaterials, which are formed of only a layer that rolls a single sheet of graphite (graphene) into a seamless molecular cylinder. Its diameter distribution and length are at the range of 0.75-3 nm and 1-50 um respectively. Multi-walled carbon nanotubes (MWCNT) are composed of more than two layers of curly graphite sheets, and they have a diameter in the range of 2-30 nm and the diameter can exceed 100 nm, with the distance between each layer being approximately 0.42 nm. Methods of functionalizing carbon nanotubes and fibrils, including SWCNT and MWCNTs, are also known. See., e.g., US20040202603 and WO1997032571A1.

Assay Methods

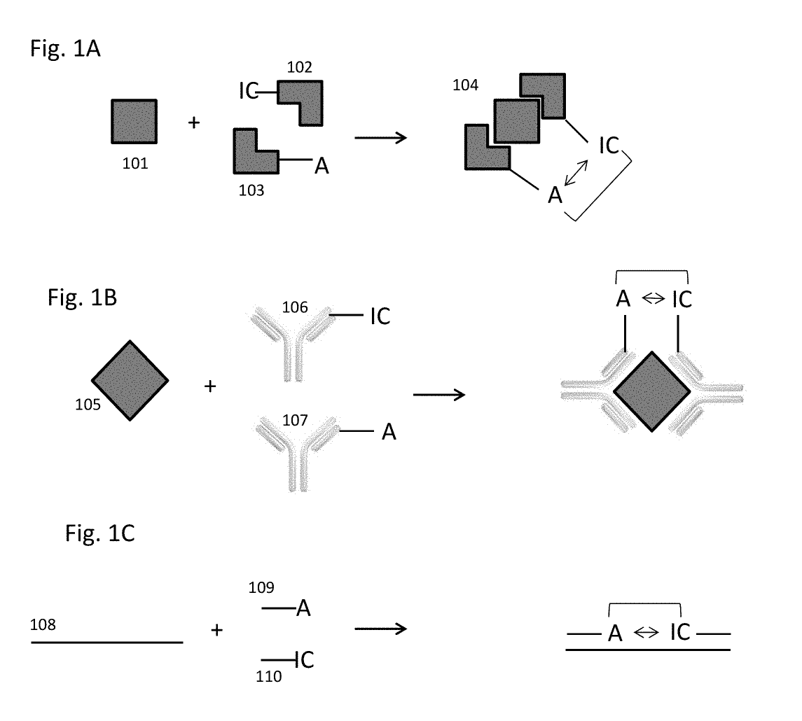

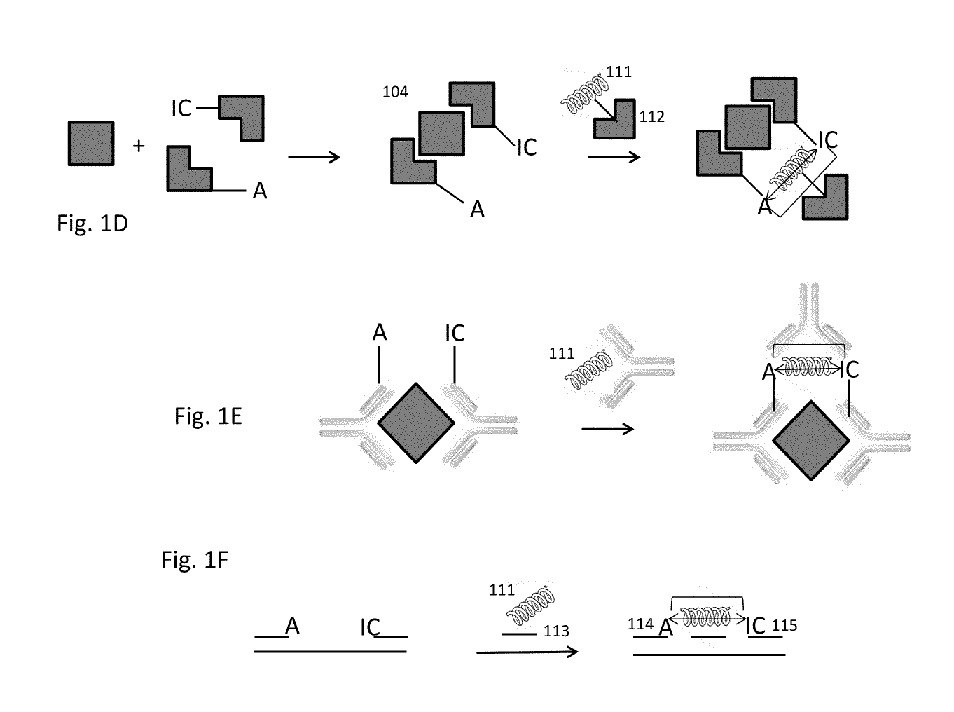

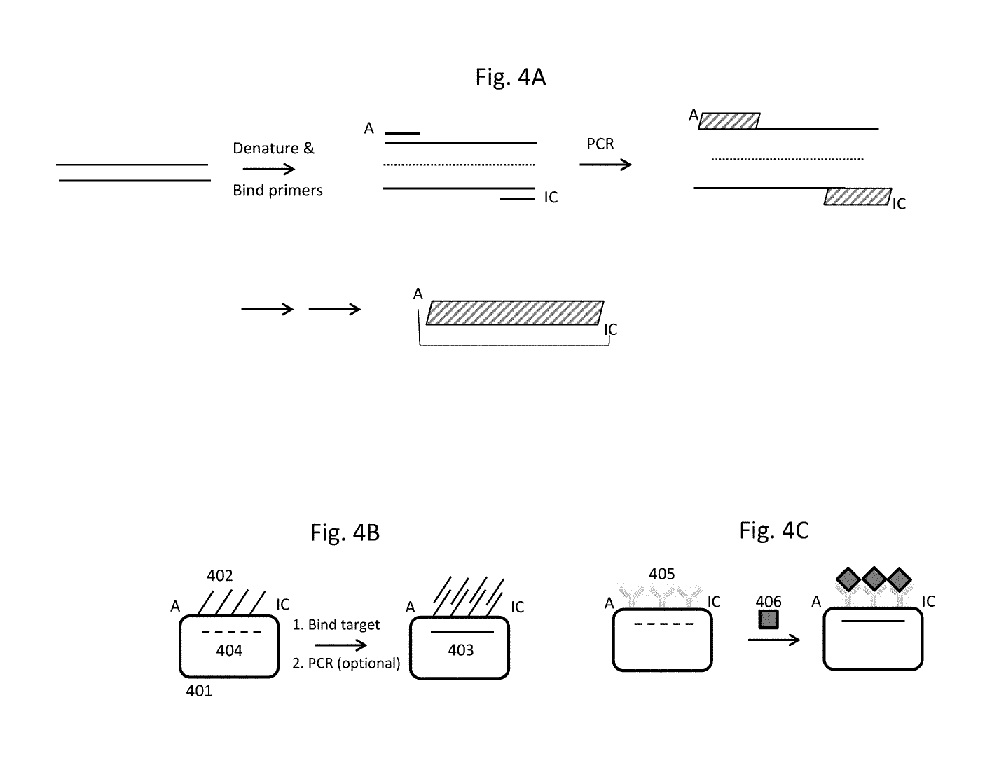

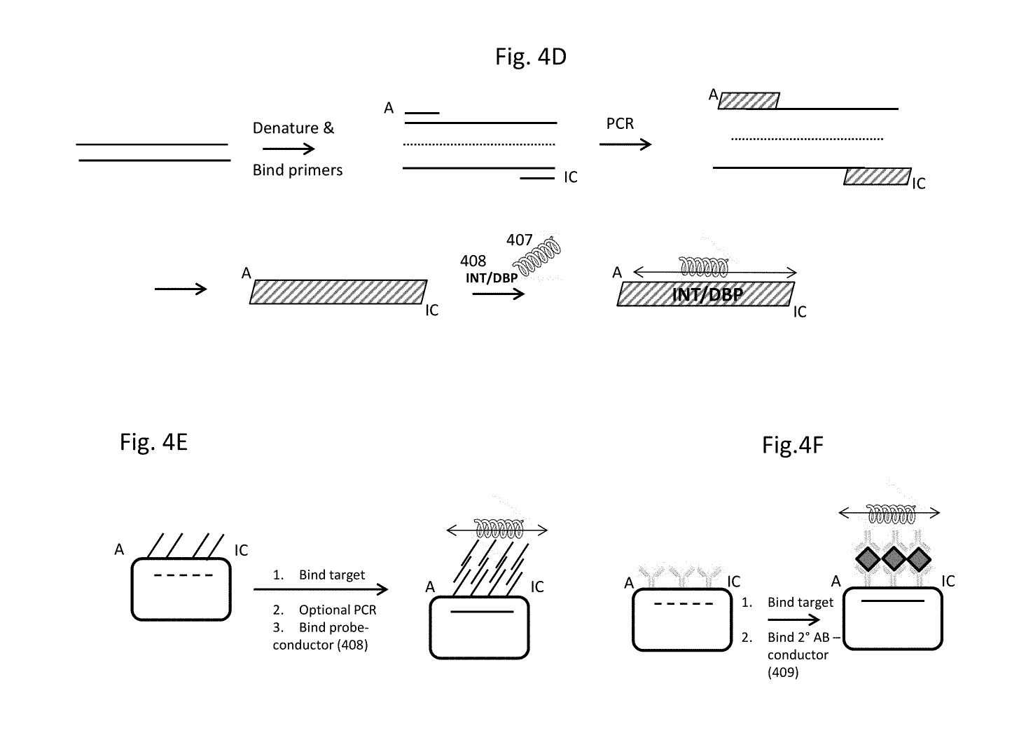

An illustration of the methods described herein is provided in FIGS. 1A-1C. In FIG. 1A, a sample comprising a target (101) is contacted with a first binding reagent (102) comprising a IC and a second binding reagent (103) comprising an antenna (A). A complex (104) is formed including the target bound to the first and second targets and the formation of the complex brings the antenna and IC in proximity to operatively connect the antenna and IC, thereby transmitting a detectable signal. The target is detected by measuring the detectable signal transmitted by the IC upon complex formation. In a specific embodiment shown in FIG. 1B, the target is an antigen (105) and the first and second binding reagents (106 and 107, respectively) are each antibodies that bind to the antigen. In another embodiment depicted in FIG. 1C, the target is an oligonucleotide sequence (108) and the first and second binding reagents (109 and 110, respectively) are each primers that bind to the 5′ and 3′ ends of the target sequence, respectively. In each of the embodiments shown in FIGS. 1A-1C the detection system is an active or passive RFID system in which the IC and antenna are provided on separate binding reagents. Alternatively, as illustrated for example in FIG. 4B, discussed in more detail below, the detection system can be a passive RFID system or a microtransponder system in which the antenna and IC are provided in a single unit on which one or more binding reagents are attached.

Specific embodiments of the assays depicted in FIGS. 1A-1C are shown in FIGS. 1D-1F, wherein a conducting species (111) is added to facilitate the operable connection between the antenna and IC. In FIG. 1D, the conducting species is linked to an additional binding reagent (112) that detects the complex (104). In FIG. 1E, the additional binding reagent is an antibody that binds the complex (104), and in FIG. 1F, the conducting species is linked to a probe (113) that binds a region of the target between the first and second primers (114 and 115, respectively). In each of the embodiments depicted in FIGS. 1A-1F, one or more of the steps can be followed by a wash step that comprises contacting the mixture with a wash reagent, e.g., a buffer or diluent, and the eluate is removed from the medium to discard any unbound species that might interfere with a subsequent detection step.

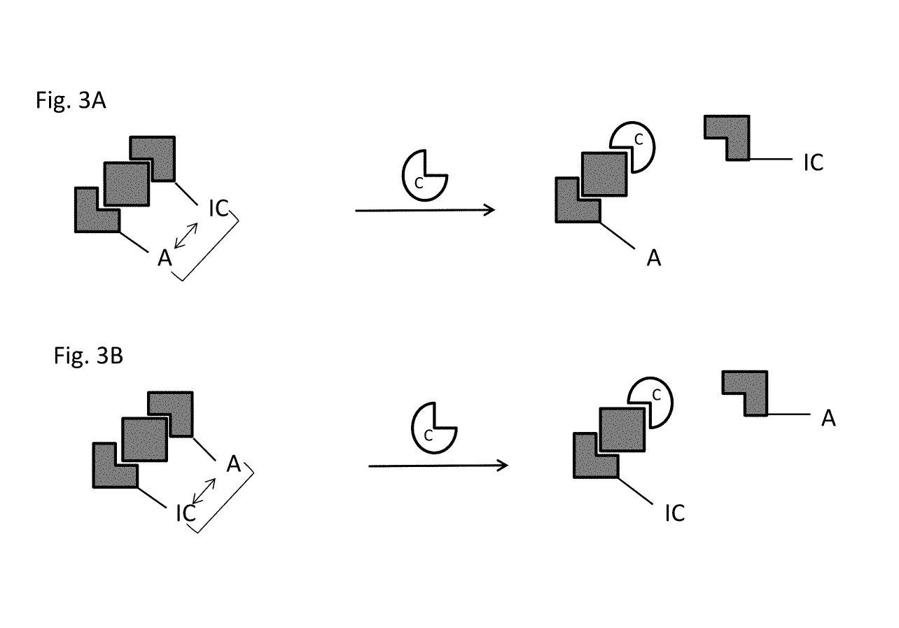

FIGS. 2A-2C illustrate another embodiment in which a target is identified by the loss of detectable signal. For example, as shown in FIG. 2A, target and the first and second binding reagents bind to form a complex 201, in which the antenna and IC are operably linked, thereby transmitting a detectable signal. The complex is then subjected to conditions sufficient to disrupt one or more binding interactions in the complex, e.g., first binding reagent to target, second binding reagent to target, and combinations thereof, and the complex is disrupted, disrupting the detectable signal (202). The complex can be disrupted by adding a competitive inhibitor, as illustrated in FIGS. 3A-3B, or alternatively, changing the pH, temperature, ionic strength, etc. One or more of the steps can be followed by an optional wash step to remove unbound, potentially interfering materials. In addition, each of the detectable complexes depicted in FIGS. 2A-2C can also be contacted with an electromagnetically conducting species that facilitates the operable connection between the antenna and IC. If a conducting species is used, a wash step is included in the method in order to remove non-specific signals between species in the matrix.

FIG. 2B illustrates a specific embodiment of the method shown in FIG. 2A wherein the complex is an immunocomplex (203). The immunocomplex is detectable upon formation but when the complex is subjected to conditions to sufficient to disrupt binding between one or more of the antibodies and the target antigen, the detectable signal is lost.

FIG. 2C illustrates yet another embodiment in which a target is detectable by measuring a loss of signal. In FIG. 2C, a double stranded oligonucleotide is denatured. A 5′-primer, 3′-primer, and an RFID labeled taqman probe are added and when the primers and probe hybridize to the target sequences, a detectable signal is transmitted. The target sequence is amplified using a nucleic acid polymerase having 5′ to 3′ nuclease activity under conditions sufficient to anneal the primers and probe to the target sequence and extend the primers, wherein the 5′ to 3′ nuclease activity of the polymerase releases a plurality of probe fragments comprising the antenna and IC, thereby quenching the detectable signal.

FIG. 4A illustrates an embodiment in which the RFID components are attached to 5′ and 3′ primers that are used in a PCR reaction that ultimately yields a complex comprising each of the extended primer sequences hybridized, thereby operably connecting the antenna and IC to transmit a detectable signal.

FIGS. 4B-4C show an embodiment in which the antenna and IC are components of a single device, e.g., a sensor (401), but they are not operably connected until the target binds to a binding reagent present on the device. In FIG. 4B, the device includes a plurality of probes (402) complementary to at least a portion of the target oligonucleotide sequences. When the target sequence binds, the circuit between the antenna and IC is complete, thereby operably connecting the antenna and IC, enabling the device to transmit a detectable signal (the operable connection is depicted in FIGS. 4B and 4C visually using a solid line (403) to reflect an operable connection between antenna and IC versus a dotted line (404) to reflect in inoperable connection). FIG. 4C illustrates the analogous system in which the device includes a plurality of antibodies (405) specific for the target analyte and upon analyte (406) binding, the circuit is complete between the antenna and IC, thereby transmitting a detectable signal.

FIGS. 4D-4F illustrate further embodiments of the method in which a conducting species (407) is used to facilitate the operable connection between the antenna and IC. In FIG. 4D, the conducting species is linked to a DNA intercalator or DNA binding protein (408) which binds to the complex, thereby facilitating the operable connection between antenna and IC. FIG. 4E is the analogue of FIG. 4B with the addition of the electromagnetically conducting species bound to a probe (408) to facilitate the operable connection, and likewise, FIG. 4F is analogous to FIG. 4C with the addition of the conducting species bound to a secondary antibody (409).

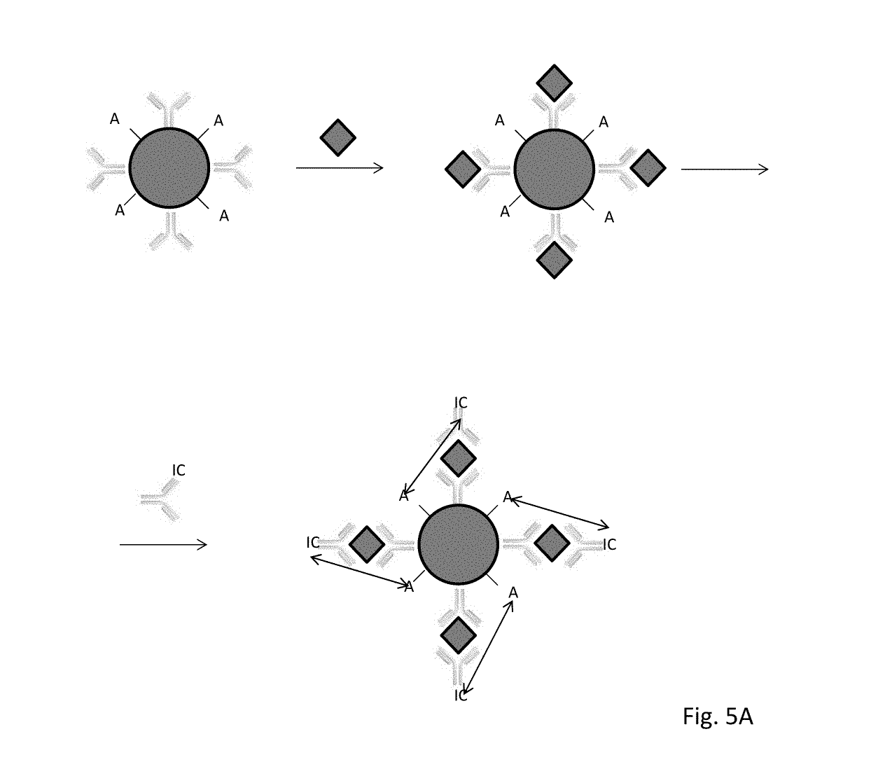

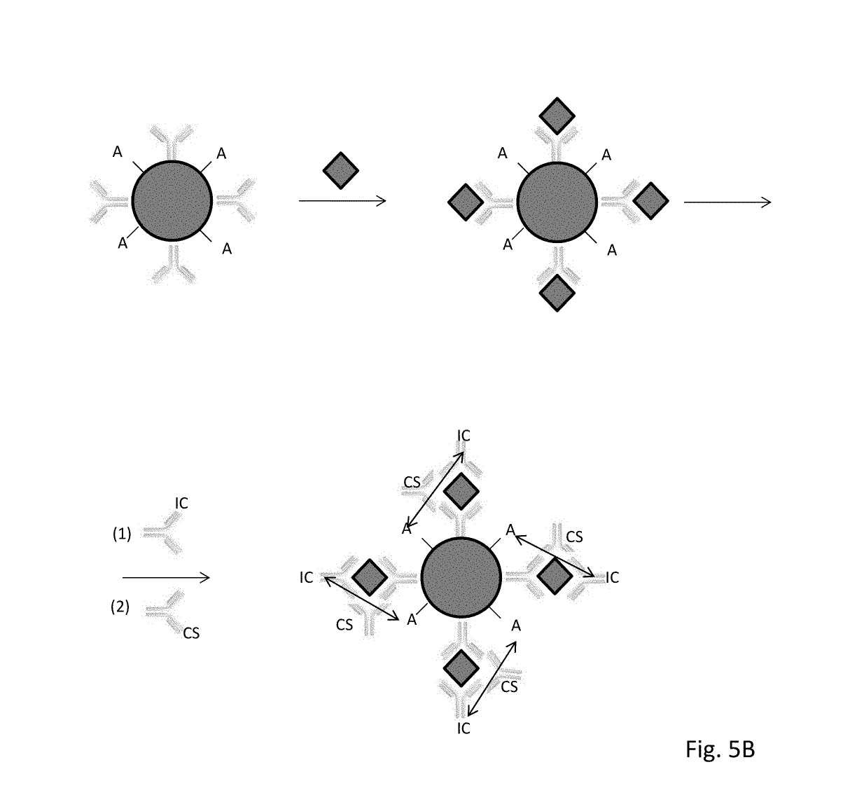

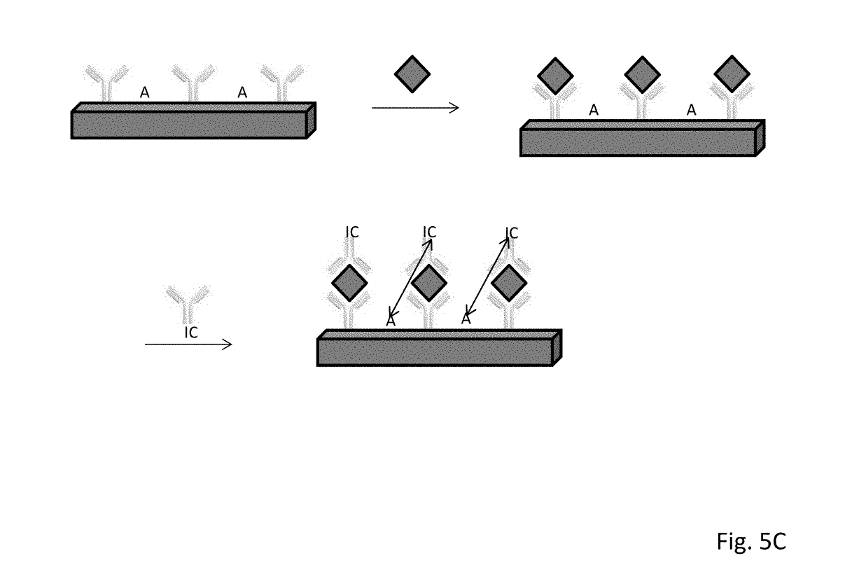

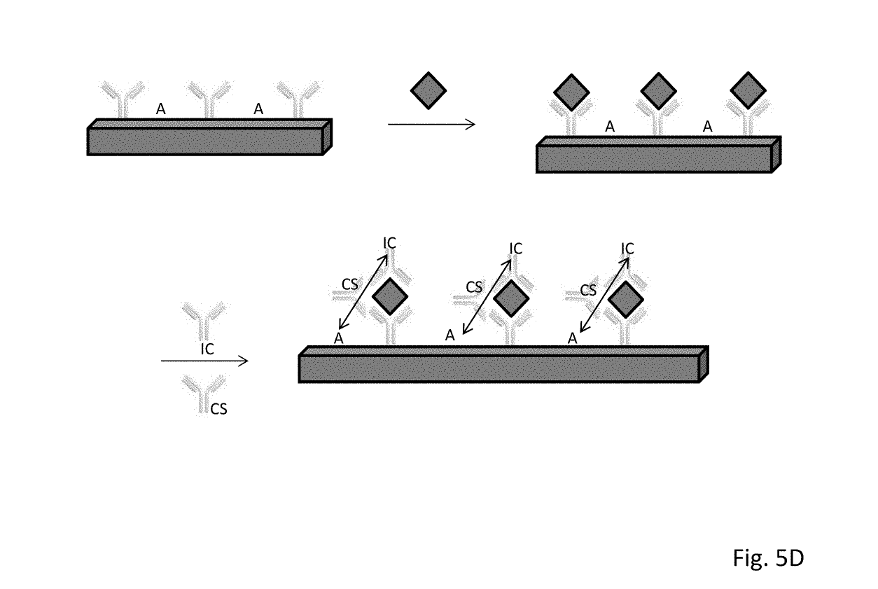

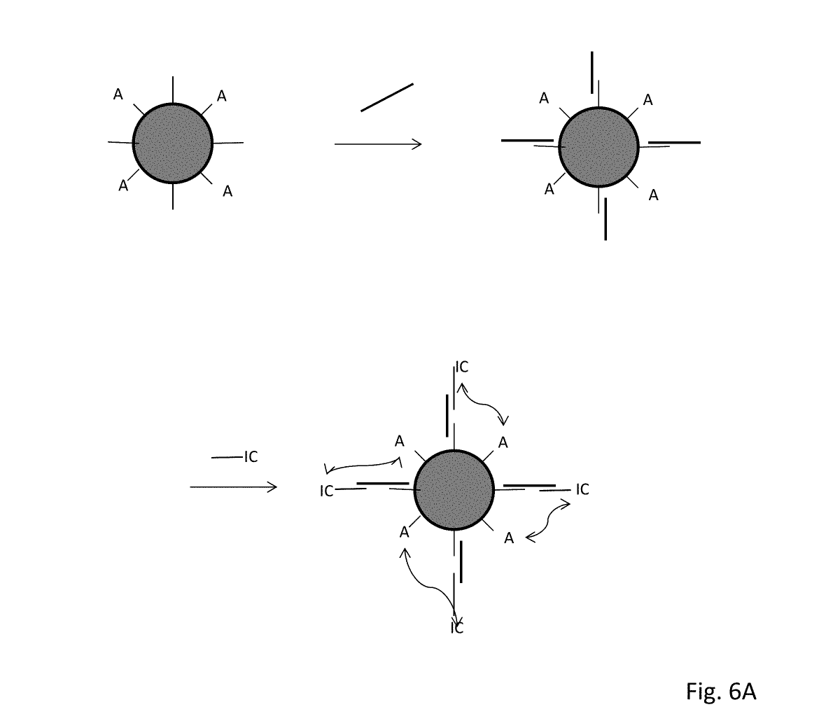

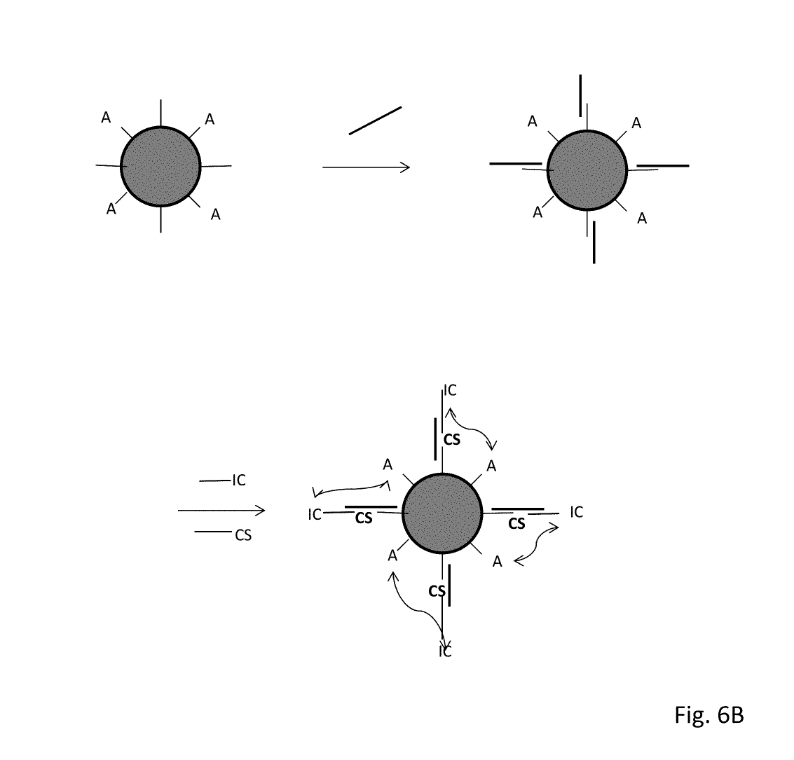

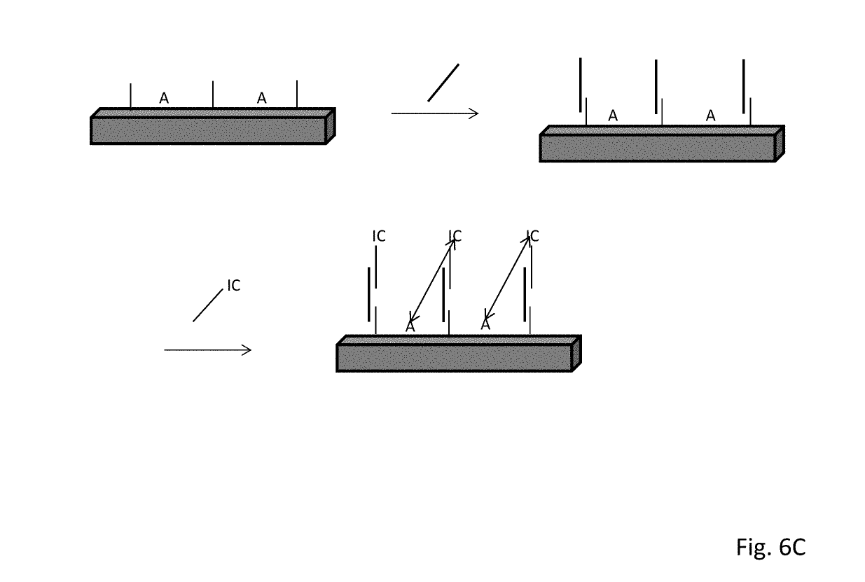

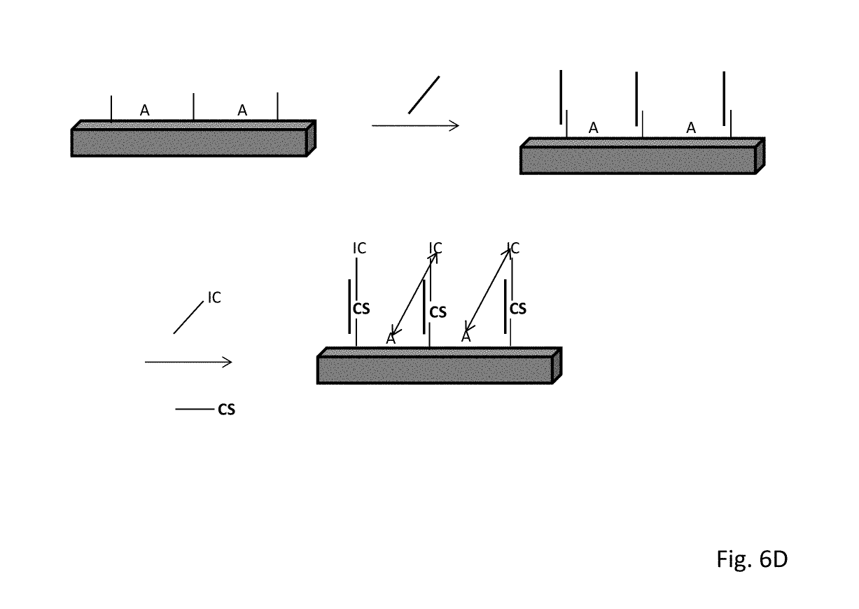

FIGS. 5A-5D and 6A-6D illustrate a further embodiment involving a solid phase as a scaffold on which one or more binding reagents and an RFID component, e.g., one or the other of an antenna or IC, are bound. The target analyte is added and then a second binding reagent is added, wherein the second binding reagent is attached to the companion RFID component, e.g., if the solid phase includes a plurality of antennas, then the second binding reagent(s) will comprise a plurality of ICs, and vice versa. When the second binding reagents are bound, the ICs and antennas are operably connected, yielding a detectable signal. FIGS. 5A and 6A illustrate embodiments in which the solid phase is a particle but the target and binding reagents are antigen/antibodies (FIG. 5A) or oligonucleotides/complements (FIG. 6A). FIGS. 5B and 6B illustrate another embodiment in which the solid phase is a lateral support and the target and binding reagents are antigen/antibodies (FIG. 5B) or oligonucleotides/complements (FIG. 6B). FIGS. 5B and 5D are specific embodiments of FIGS. 5A and 5C, and likewise, FIGS. 6B and 6D are specific embodiments of FIGS. 6A and 6C, wherein a conducting species is added to facilitate the operable connection between antenna and IC.

Certain (but not all) detection methods described herein may benefit from or require a wash step to remove unbound components (e.g., one or more of the binding species, conducting species, unbound target, etc.). Accordingly, the methods described herein may include one or more wash steps.

A wide variety of solid phases/surfaces are suitable for use in the methods described herein including conventional surfaces from the art of binding assays. Solid phases/surfaces may be made from a variety of different materials including polymers (e.g., polystyrene and polypropylene), ceramics, glass, composite materials (e.g., carbon-polymer composites such as carbon-based inks). Suitable surfaces include the surfaces of macroscopic objects such as an interior surface of an assay container (e.g., test tubes, cuvettes, flow cells, FACS cell sorter, cartridges, wells in a multi-well plate, etc.), slides, assay chips (such as those used in gene or protein chip measurements), pins or probes, beads, filtration media, lateral flow media (for example, filtration membranes used in lateral flow test strips), etc.

Suitable solid phases/surfaces also include particles (including but not limited to colloids or beads) commonly used in other types of particle-based assays e.g., magnetic, polypropylene, and latex particles, materials typically used in solid-phase synthesis e.g., polystyrene and polyacrylamide particles, and materials typically used in chromatographic applications e.g., silica, alumina, polyacrylamide, polystyrene. The materials may also be a fiber such as a carbon fibril. Microparticles may be inanimate or alternatively, may include animate biological entities such as cells, viruses, bacterium and the like. A wide variety of different types of particles that may be attached to binding reagents are sold commercially for use in binding assays. These include non-magnetic particles as well as particles comprising magnetizable materials which allow the particles to be collected with a magnetic field. In one embodiment, the particles are comprised of a conductive and/or semiconductive material, e.g., colloidal gold particles. The microparticles may have a wide variety of sizes and shapes. By way of example and not limitation, microparticles may be between 5 nanometers and 100 micrometers. Preferably microparticles have sizes between 20 nm and 10 micrometers. The particles may be spherical, oblong, rod-like, etc., or they may be irregular in shape.

Optionally, particles may be coded to allow for the identification of specific particles or subpopulations of particles in a mixture of particles. The use of such coded particles has been used to enable multiplexing of assays employing particles as solid phase supports for binding assays. In one approach, particles are manufactured to include one or more fluorescent dyes and specific populations of particles are identified based on the intensity and/or relative intensity of fluorescence emissions at one or more wave lengths. This approach has been used in the Luminex xMAP systems (see, e.g., U.S. Pat. No. 6,939,720) and the Becton Dickinson Cytometric Bead Array systems. Alternatively, particles may be coded through differences in other physical properties such as size, shape, imbedded optical patterns and the like. One or more particles provided in a mixture or set of particles may be coded to be distinguishable from other particles in the mixture by virtue of particle optical properties, size, shape, imbedded optical patterns and the like.

Optionally, the solid phase/surface may comprise one or more boundaries of a container, including but not limited to a flow cell, one or more wells of a multi-well plate, tube, cuvette, assay container, slide, chips (such as those used in gene or protein chip measurements), pin, probe, or lateral flow media.

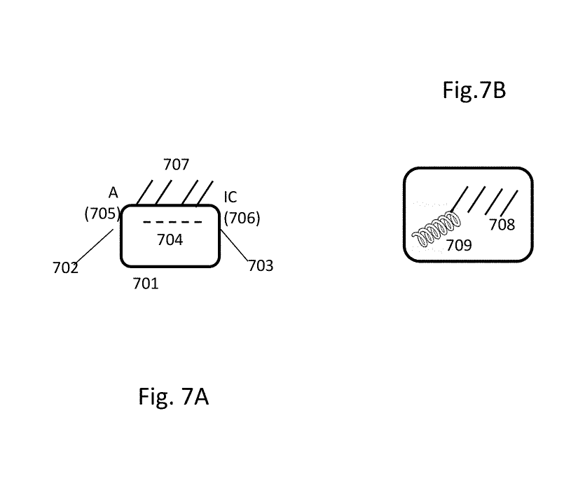

A specific embodiment of a suitable solid phase for use in the methods described herein is illustrated in FIG. 7A-7B. A sensor (701) having a proximate end (702), a distal end (703), and a gate terminal (704) spanning the proximate and distal end. An RFID antenna (705) is affixed to the proximate end and an RFID IC (706) associated with the RFID antenna is affixed to the distal end, and a plurality of first binding reagents (707) capable of binding a target are bound to the gate terminal. Also illustrated in the FIG. 7B are additional components used with the sensor, e.g., in a kit, including, in one or more separate containers, vials, or compartments, a plurality of second binding reagents (708) capable of binding the target, wherein at least one of said plurality of second binding reagents is linked to a conducting species (709). In the embodiments shown in FIGS. 7A-7B, the binding reagents are primers for a target nucleic acid sequence, but the binding reagents can also be any suitable alternative binding reagent, e.g., an antibody, antigen, receptor, ligand, hapten, epitope, mimitope, or aptamer.

Also provided is a sensor comprising a proximate end, a distal end, and a gate terminal spanning the proximate and distal end, an RFID antenna affixed to the proximate end and an RFID IC associated with the RFID antenna affixed to the distal end, and a plurality of first binding reagents capable of binding a target bound to the gate terminal. As described herein, the first binding reagent included with the sensor includes but is not limited to, any molecule known in the art to be capable of, or putatively capable of, binding an analyte of interest (a target). An analyte of interest may include but is not limited to, e.g., a whole cell, a subcellular particle, virus, prion, viroid, nucleic acid, protein, antigen, lipoprotein, lipopolysaccharide, lipid, glycoprotein, carbohydrate moiety, cellulose derivative, antibody or fragment thereof, peptide, hormone, pharmacological agent, cell or cellular components, organic compounds, non-biological polymer, synthetic organic molecule, organo-metallic compounds or an inorganic molecule present in the sample. Thus, the binding reagents include but are not limited to receptors, ligands for receptors, antibodies or binding portions thereof (e.g., Fab), proteins or fragments thereof, nucleic acids, oligonucleotides, primers, probes, glycoproteins, polysaccharides, antigens, epitopes, cells and cellular components, subcellular particles, carbohydrate moieties, enzymes, enzyme substrates, lectins, protein A, protein G, organic compounds, organometallic compounds, viruses, prions, viroids, lipids, fatty acids, lipopolysaccharides, peptides, cellular metabolites, hormones, pharmacological agents, tranquilizers, barbiturates, alkaloids, steroids, vitamins, amino acids, sugars, nonbiological polymers, biotin, avidin, streptavidin, organic linking compounds such as polymer resins, lipoproteins, cytokines, lymphokines, hormones, synthetic polymers, organic and inorganic molecules, etc. Nucleic acids and oligonucleotides can refer to DNA, RNA and/or oligonucleotide analogues including but not limited to: oligonucleotides containing modified bases or modified sugars, oligonucleotides containing backbone chemistries other than phosphodiester linkages, and/or oligonucleotides, that have been synthesized or modified to present chemical groups that can be used to form attachments to (covalent or non-covalent) to other molecules. In a specific embodiment of the sensor, the binding reaction evaluated using the sensor is a nucleic acid binding reaction wherein the target is an oligonucleotide and the first binding reagent is sequence complementary to the 5′ and/or 3′ ends of the target oligonucleotide sequence, respectively. In another specific embodiment of the sensor, the binding reaction evaluated using the sensor is an immunoassay, the target is a protein antigen, and the first binding reagent an antibody that binds to different epitopes of the antigen.

Also contemplated is a kit comprising a sensor as described herein and, in one or more separate containers, vials, or compartments, a plurality of second binding reagents capable of binding the target, wherein at least one of said plurality of second binding reagents is linked to a conducting species. The second binding reagent can be any of the binding reagent species identified herein. The conducting species comprises carbon fibrils, carbon nanotubes, graphitic nanotubes, graphitic fibrils, carbon tubules, fibrils, and buckeytubes. Alternatively, the conducting species can include ferrocene and/or ferrocene derivatives. In a particular embodiment, the conducting species is a carbon nanotube. The kit can also include one or more additional binding reagents, as defined hereinabove, buffers, diluents, PCR Master Mix and reagents required for isothermal amplification, including but not limited to, DNA polymerase, e.g., Taq polymerase, dNTPs, MgCl2, helicase, nicking enzyme, etc.; as well as reagents used in an immunoassay, e.g., antibodies or fragments thereof, antigens, bovine serum albumin, streptavidin, avidin, biotin, labeled assay components, e.g., components including a radiolabel, chemiluminescent label, electrochemiluminescent, or luminescent label, fluorophore, etc., immunoassay coreactants, e.g., tertiary amines (if the assay is an electrochemiluminescent assay, a coreactant including tripropyl amine is used in the assay), etc.

The kit can also include any suitable vessel or container used to conduct the assay, including but not limited to assay container (e.g., test tubes, cuvettes, flow cells, FACS cell sorter, cartridges, wells in a multi-well plate, etc.), slides, assay chips (such as those used in gene or protein chip measurements), pins or probes, beads, filtration media, lateral flow media (for example, filtration membranes used in lateral flow test strips), etc.

Reagent/Component Information Stored to Non-Volatile Memory

In the embodiments described herein and illustrated in the accompanying Figures, the RFID IC comprises information about the assay and the assay components which can be read once the detectable signal is transmitted. The system reads the component information stored to the RFID and that information is used by the system to identify the components. The system optionally reviews the component information stored locally on the system in the local storage medium to identify that information stored to the storage medium that can be used for the conduct of an assay using a given component. If the storage medium includes the information for that component, the system will commence running an assay. If the storage medium does not include information for those particular components, the system can query the user for that component information and the user can communicate with the vendor to receive the requisite information, e.g., via email, compact diskette, memory card/stick, flash drive, web data storage service, etc. The vendor sends component information binary files (including but not limited to encrypted XML files) to the user, e.g., as an email attachment to a user email account, the user loads that file attachment to the assay system and the system software stores the component information to the local system component information repository. The components can then be used in the system.

In an alternative embodiment, the database can be connected to the system via a direct interface which can automatically obtain the component information from the database if it is not available on the system locally. Thereafter the system software queries the system data repository for the component information associated with that RFID and if that component information is available locally on the system, the software will adjust the system based on the component information, if necessary. If the component information is not present in the local system data repository, the system will either (i) prompt the user to manually obtain the component information from the vendor, or (ii) automatically, via a direct interface with the remote database, obtain the component information from the remote database and store that information locally on the system data repository. Once the component information is available locally on the system, the software adjusts the system based on the component information, if necessary, and conducts an assay.

The system can adjust the assay parameters prior to initiating an assay based on the information saved to the RFID and/or stored or provided via a direct or indirect interface. Thereafter, the system makes the appropriate electrical, fluidic and/or optical connections (making use of electrical, fluidic and/or optical connectors on the consumable and system) and conducts an assay using the components. The assay can also involve adding one or more assay reagents to a component, e.g., a reaction vessel, and instructions for adding those various assay reagents can be saved to the RFID and/or provided as component information and the system adds those reagents to the component before or during the assay according to the instructions saved to the component identifier and/or provided as component information.

The methods described herein are conducted in an assay system that is operably connected to a storage medium including a data repository comprising one or more assay protocol scripts. The system is also operably associated with a reader adapted to read information from an RFID. In one embodiment, the storage medium, data repository, and reader are components of the assay system. Alternatively, at least the storage medium and/or the data repository can be remotely connected to the system, e.g., over a computer network. The reader can be an internal or external component of the system. In one embodiment, the assay system is pre-programmed to identify the assay protocol that will be used by the system and the system queries the data repository to identify the associated requirement list for that assay protocol. Alternatively, the system can identify an assay protocol based on the sample and/or reagent information read from the identifiers and query the data repository for the associated requirement list after the identifiers have been read by the reader.

In an additional embodiment, one or more vessels or containers used to store or house samples or reagents can include supplemental identifiers, e.g., additional RFID labels, bar codes, EEPROM, or combinations thereof. For example, the assay or system may manipulate samples or reagents in a one or more test tubes, flasks, microwell or microtitre plates and each vessel or container can include a supplemental identifier that uniquely identifies that vessel. The reader associated with the assay system can read the information stored to each of the RFID labels and supplemental identifiers and compare that information to the information stored to the data repository. In a specific embodiment, the sample and one or more reagents are uniquely labeled using RFID labels and consumables used in the conduct of an assay, e.g., test tubes, flasks, a microwell plate or reaction chip, are labeled with a supplemental identifier, e.g., a bar code or RFID.

Sample, Reagent and/or Consumable Information

The identifiers (e.g., RFID tags and/or microtransponders) include non-volatile memory that can be programmed with information which can be used before, during or after an assay or a step of a multi-step assay to control the operation of the assay system or a subsystem thereof. The terms “sample information,” “reagent information,” and “consumable information” can include any information used to uniquely identify a particular reagent, sample, or consumable or to distinguish a reagent, sample, or consumable from other components in the system. “Component information” is also used herein to refer to any sample, reagent, or consumable information that is not defined by the type of component.

### Component Information

Component information can include but is not limited to component type, component identification information, the date of manufacture, lot number, expiration date, assay names and/or identifiers, information concerning assay quality control, calibration information such as a master calibration curve, the number and names of assay calibrators and/or assay calibrator acceptance ranges, clinical trial information, formulation information, the identity of and/or results obtained from diagnostic tests performed on the component, supplier information, lot identification information, lot specific analysis parameters, manufacturing process information, raw materials information, expiration date, Material Safety Data Sheet (MSDS) information, product insert information (i.e., any information that might be included or described in a product insert that would accompany the component, e.g., the assay type, how the assay is performed, directions for use of the component, etc.), and/or threshold and/or calibration data for a component.

Component information can also relate to chain of custody, e.g., information regarding the control, transfer and/or analysis of the sample, reagent, and/or an assay consumable. Chain of custody information can be selected from customer identification, sample identification, time and date stamp for an assay, custody and/or location information for the component before and after the conduct of the assay, assay results for a given sample, as well as customer created free text comments input before, during or after an assay is processed by the system using that component. Still further, chain of custody information can include time, date, manufacturing personnel or processing parameters for one or more steps during the manufacture of the component, custody, location and/or storage conditions for the component following manufacture and/or between steps during the manufacture of the component.

Still further, component information can be used as a security mechanism, e.g., to confirm that the correct reagent, sample, or consumable is being used in the system. The information can include a digital signature to prove that the component was manufactured by the designated vendor. In one embodiment, if an inappropriate consumable is present in the system, e.g., a counterfeit consumable or a consumable that is otherwise incompatible with the assay system, the controller will disable the system, reader or a subsystem thereof. In addition or alternatively, the information can be used to detect the proper placement of an assay consumable in the system, e.g., the proper orientation of the assay consumable or a portion thereof, in the assay system, such that the controller will disable the system, reader or a component thereof until the assay consumable is placed in the correct orientation. Still further, the information can also be used to detect a defect in the assay consumable or an assay test site and/or domain and the controller will disable the system, reader or a component thereof accordingly. In a further embodiment, the component can be subjected to a quality control process during or after its manufacture and the results of that quality control analysis can be written to the identifier for later use and/or verification by the customer of the component in an assay reader.

The component information can also include authorization information for samples, reagents, and/or consumables or test site and/or domain thereof, such as information regarding whether a particular customer has a valid license to use a particular component, including the number of times the customer is permitted to use the particular component in a particular assay and the limitations, if any, on that use, e.g., whether the customer's license is for research purposes only. Such information can also include validation information regarding whether a particular component has been subject to a recall or has otherwise become unsuitable or unauthorized for use. The recall information and an optional last recall check date and/or timestamp can be written to the identifier and/or provided as information.

The component information can further include information regarding the origin of a biological reagent used in a component, test site and/or domain, including for example an identification of an original sample from which it was derived or the number of generations removed it is from an original sample. For example, if an assay reagent used in an assay is an antibody, the information can include the identification of the hybridoma from which the antibody was derived, e.g., the ATCC accession number for that hybridoma.

According to various embodiments, biological samples or reagents that are provided in or with the consumables described above can be licensed separately from systems designed to operate on the biological reagents. In various embodiments the assay system, reader or a component thereof is coupled to a network that allows the system to communicate over public and/or private networks with computer systems that are operated by or on behalf of the customers, manufacturers and/or licensors of the biological reagents, consumables or systems. In various embodiments, a limited license can provide for the use of licensed biological reagents, consumables or systems for a particular biological analysis on only licensed systems. Accordingly, a system can authenticate a biological reagent, consumable or system based on, for example, a digital signature contained in the identifier associated with a particular consumable and/or provided as information, if a particular customer has a valid license. In various embodiments, the identifier and/or information can also be used to provide for a one time use such that biological reagents cannot be refilled for use with the same authentication.

In certain embodiments, when the identifier is read by a system, reader or component thereof that has access to a public or private data network operated by or on behalf of the customers, manufacturers and/or licensors of the biological reagents, consumables or systems, certain information can be communicated to the assay system and read, written or erased locally via the identifier/controller on the assay system. For example, recall and/or license information can be a subset of information that is available via a direct and/or indirect interface, whereas additional information e.g., lot-specific, expiration date, calibration data, component specific information, assay results information, component security information, or combinations thereof, can be stored locally on the identifier and otherwise unavailable via the network connections on the assay system. In one embodiment, recall, license and/or component security information can be available via the network connections on the assay system and/or stored to the storage medium as information and the remaining information is stored locally on the identifier. The assay system or reader includes system hardware, system firmware, system data acquisition and control software, and method or information. In various embodiments, the system hardware includes electronic control and data processing circuitry, such as a microprocessor or microcontroller, memory, and non-volatile storage. In various embodiments, the system hardware also includes physical devices to manipulate biological reagents such as robotics and sample pumps. In various embodiments, the system firmware includes low-level, computer-readable instructions for carrying out basic operations in connection with the system hardware. In various embodiments, the system firmware includes microprocessor instructions for initializing operations on a microprocessor in the system hardware.

In addition, the component information can include assay process information concerning the individual assay parameters that should be applied by the system during an assay using that component. For example, such information can include a sequence of steps for a given assay, the identity, concentration and/or quantity of assay reagents that should be used or added during the assay or during a particular step of an assay, e.g., buffers, diluents, and/or calibrators that should be used in that assay. The information can also include the type or wavelength of light that should be applied and/or measured by the system during the assay or a particular step of a multi-step assay; the temperature that should be applied by the system during the assay; the incubation time for an assay; and statistical or other analytical methods that should be applied by the system to the raw data collected during the assay.

In one embodiment, one or more steps of an assay protocol can be tailored to an individual component or lot of components. One or more steps of a protocol can differ from component lot to lot and/or from individual component to component within a given lot and the information stored to the system includes instructions to tailor those steps of the assay protocol. This type of information can be used by the system to adjust one or more operations performed by the system before, during and/or after the conduct of an assay by the system. Moreover, this type of information can optionally be adjusted by the system user at the user's discretion. For example, dilution steps in an assay protocol can be adjusted to account for lot to lot or component to component differences. The amount of diluent added and/or the nature of the diluent can be altered based on such differences. Similarly, the amount of a given reagent that can be added during the conduct of an assay, an incubation period and/or temperature for one or more steps of an assay can also be dependent on lot to lot or component to component differences. Each of these is a non-limiting example of information that can be saved to the storage medium of the system.

Moreover, the information comprises information that directly or indirectly controls a component of the assay system, e.g., one or more photodetectors, a light tight enclosure; mechanisms to transport the component into and out of the system; mechanisms to align and orient the components with the one or more subsystem(s); additional mechanisms and/or data storage media to track and/or identify components, mechanisms to transfer, store, stack, move and/or distribute one or more components; mechanisms to detect signal from a consumable during the assay sequentially, substantially simultaneously or simultaneously from a plurality of test sites of the consumable; or combinations thereof.

The information can also include assay process information comprising assay parameters to be applied by the system during the assay; a sequence of steps to be applied by the system during the assay; the identity, concentration, and/or quantity of assay reagents to be used or added during the assay; the temperature to be applied by the system during the assay; an incubation time for the assay; statistical or analytical methods to be applied by the system to raw data collected during the assay; or combinations thereof (such assay process information can optionally be adjusted by the user). In one specific embodiment, the assay conducted with the consumable is a multi-step assay and the assay process information relates to a step or step(s) of the multi-step assay.

In addition, a given assay protocol can require a set of components of a particular type. Therefore, if the user inputs a specific type of component, e.g., a multi-well assay plate, for use in a particular assay protocol, one or more additional components can be required to carry out that assay protocol in the system, e.g., one or more reagents can be required for use with that multi-well assay plate. Each of the required components can include an identifier with information concerning the component requirements for an assay protocol. When one of the required components is input into the assay system and the reader interacts with the identifier for that component, the system will take an inventory of the components present in the system and compare the results to the requirements list stored to the identifier and/or stored to the storage medium and/or provided as information. If any required components are not present or are present in insufficient supply, the system will prompt the user to input the additional required components for that assay protocol.

In another embodiment, the component information further includes one or more analytical tools that can be applied by the system to analyze data generated during and/or after the conduct of an assay. In addition, such analytical tools can include instructions for the user and/or the system to generate a specific output by the system software after the conduct of an assay, e.g., a tailored data report and/or format for the results of the analysis based on the information. Alternatively or additionally, the analytical tools can further include one or more statistical algorithms that can be applied by the system to the data. For example, the component information can include a selection of two or more statistical algorithms that can be used to analyze data resulting from use of a given component and the user can optionally select the appropriate algorithm for the desired data analysis. The information can also include information that can be used by the user to select the appropriate algorithm for his or her needs, e.g., technical notes or literature references related to algorithm selection.

Analytical tools can differ from component lot to lot and/or from individual component to component within a given lot. In this embodiment, the information is used by the system to adjust the analytical processing tools applied by the system software in the conduct of an assay or after the assay is completed and the results are generated and/or displayed. Such analytical processing tools include but are not limited to assay thresholds and/or calibration curves that can be applied to one or more steps of an assay protocol that can also be altered based on component differences. In a specific embodiment, for a given component type and/or desired use, the information can include a project management tool that schedules the conduct of one or more assays or steps thereof using a given component in the system or with a set of components. Still further, such analytical processing tools can optionally be adjusted by the system user at the user's discretion. Analytical tools can be sent to the user via a direct or indirect interface between the system and the user.

### Reagent Information

Reagent information can include but is not limited to reagent type, formulation, the date of manufacture, lot number, expiration date, reagent chain of custody information, associated assay names and/or identifiers, information concerning reagent quality control, calibration information such as a master calibration curve, the number and names of assay calibrators and/or assay calibrator acceptance ranges, supplier information, lot identification information, lot specific analysis parameters, manufacturing process information, raw materials information, expiration date, Material Safety Data Sheet (MSDS) information, product insert information (i.e., any information that might be included or described in a product insert that would accompany the reagent, e.g., the assay type, how the assay is performed, directions for use of the reagent, etc.), and/or threshold and/or calibration data for a reagent.

### Sample Information

Sample information can include sample type, patient identification information, clinical trial information (i.e., information about a clinical trial for which the sample has been collected), sample collection information, sample chain of custody information, sample formulation information, the identity of and/or results obtained from additional diagnostic tests performed on the sample, and combinations thereof.

The present application is not to be limited in scope by the specific embodiments described herein. Indeed, various modifications in addition to those described herein will become apparent to those skilled in the art from the foregoing description and accompanying figures. Such modifications are intended to fall within the scope of the claims. Various publications are cited herein, the disclosures of which are incorporated by reference in their entireties.

What is claimed:

1. A method of detecting a target comprising
a. Contacting a sample comprising said target with a sensor comprising (i) an RFID antenna, (ii) an RFID IC associated with said RFID antenna, and (iii) a plurality of first binding reagents capable of binding said target, thereby forming a target-bound complex;
b. Mixing said target-bound complex with a plurality of second binding reagents capable of binding said target and thereby forming a detectable target-bound complex, wherein one or more second binding reagents are linked to a conducting species that operably connects the RFID antenna and IC upon formation of said detectable target-bound complex to produce a detectable signal;
c. Detecting said target by measuring said detectable signal; and optionally,
d. Reading sample and/or reagent information stored to said RFID IC.

2. The method of claim 1 wherein said conducting species comprises carbon fibrils, carbon nanotubes, graphitic nanotubes, graphitic fibrils, carbon tubules, fibrils, and buckeytubes.

3. The method of claim 1 wherein the conducting species comprises a carbon nanotube.

4. The method of claim 1 wherein said target is a nucleic acid and each of said first and second binding reagents are nucleic acid probes each comprising a nucleic acid sequence complementary to said target.

5. The method of claim 1 wherein said sensor comprises a particle.

6. The method of claim 5 wherein said sensor is a magnetic particle.

7. The method of claim 1 wherein said sensor comprises polypropylene, latex, polystyrene, polyacrylamide, silica, alumina, fiber, carbon fiber, graphite or graphene.

8. The method of claim 1 wherein said IC comprises non-volatile memory including information related to said method and one or more components used in said method.

9. The method of claim 1 wherein said information comprises target and reagent information.

10. The method of claim 1 wherein said information comprises a protocol for the formation of said detectable target-bound complex.

11. The method of claim 1 wherein said method further comprises reading said information from said IC and adjusting one or more steps of an assay protocol based on said information.

12. The method of claim 1 wherein said antenna is tuned to a unique resonant frequency.

13. The method of claim 1 wherein said method further comprises washing the mixture formed after one or more of steps (a) and (b).

14. A sensor comprising a proximate end, a distal end, and a gate terminal spanning the proximate and distal end, an RFID antenna affixed to the proximate end and an RFID IC associated with the RFID antenna affixed to the distal end, and a plurality of first binding reagents capable of binding a target bound to the gate terminal.

15. A kit comprising a sensor of claim 14 and, in one or more separate containers, vials, or compartments, a plurality of second binding reagents capable of binding the target, wherein at least one of said plurality of second binding reagents is linked to a conducting species.
